SEQUENCE LISTING (93 sequences)

SEQ ID NO: 1 - DNA, 30 bp, Artificial Sequence
  Oligonucleotide
cgggctagcacacctcaaaatattactgat

SEQ ID NO: 2 - DNA, 28 bp, Artificial Sequence
  Oligonucleotide
ggcgaattcatttgccatactaattgcg

SEQ ID NO: 3 - DNA, 31 bp, Artificial Sequence
  Oligonucleotide
cggtctagagacacctcaaaatattactgat

SEQ ID NO: 4 - DNA, 32 bp, Artificial Sequence
  Oligonucleotide
aaaaagcttttagtctctgtctctgccgccac

SEQ ID NO: 5 - DNA, 28 bp, Artificial Sequence
  Oligonucleotide
ggcgtcgacatttgccatactaattgcg

SEQ ID NO: 6 - DNA, 30 bp, Artificial Sequence
  Oligonucleotide
ggcaagcttttaaagcttgttaaaagcctt

SEQ ID NO: 7 - protein, 310 aa, Vibrio cholerae
CACACCTCAAAATATTACTGATTTGTGTGCAGAATACCACAACACACAAATACATACGCTAAATGATAAGATATTTTCGTATACAGAATCTCTAGCTGGAAAAAGAGAGATGGCTATCATTACTTTTAAGAATGGTGCAACTTTTCAAGTAGAAGTACCAGGTAGTCAACATATAGATTCACAAAAAAAAGCGATTGAAAGGATGAAGGATACCCTGAGGATTGCATATCTTACTGAAGCTAAAGTCGAAAAGTTATGTGTATGGAATAATAAAACGCCTCATGCGATTGCCGCAATTAGTATGGCAAAT

SEQ ID NO: 8 - protein, 474 aa, Arachis hypogaea
ATGGGCGTCTTCACTTTCGAGGATGAAATCACCTCCACCGTGCCTCCGGCCAAGCTTTACAATGCTATGAAGGATGCCGACTCCATCACCCCTAAGATTATTGATGACGTCAAGAGTGTTGAAATTGTTGAGGGAAACGGTGGTCCCGGAACCATCAAGAAACTCACCATTGTCGAGGATGGAGAAACCAAGTTTATCTTGCACAAGGTGGAGTCAATAGATGAGGCCAACTATGCATACAACTACAGCGTTGTTGGAGGAGTGGCTCTGCCTCCCACGGCGGAGAAGATAACATTTGAGACAAAGCTGGTTGAAGGACCCAACGGAGGATCCATTGGGAAGCTTACTCTCAAGTACCACACCAAAGGAGATGCAAAGCCAGATGAGGAAGAGTTGAAGAAGGGTAAGGCCAAGGGTGAAGGTCTCTTCAGGGCTATTGAGGGTTACGTTTTGGCCAACCCTACTCAATATTAG

SEQ ID NO: 9 - DNA, 29 bp, Artificial Sequence
  Oligonucleotide
aaagtcgacatgggcgtcttcactttcga

SEQ ID NO: 10 - DNA, 30 bp, Artificial Sequence
  Oligonucleotide
ggcgcggccgcctaatattgagtagggttg

SEQ ID NO: 11 - DNA, 30 bp, Artificial Sequence
  Oligonucleotide
cgagaattcacacctcaaaatattactgat

SEQ ID NO: 12 - DNA, 25 bp, Artificial Sequence
  Oligonucleotide
cgagtcgacattgccatactaattg

SEQ ID NO: 13 - DNA, 29 bp, Artificial Sequence
  Oligonucleotide
cgctctagacacacctcaaaatattactg

SEQ ID NO: 14 - DNA, 30 bp, Artificial Sequence
  Oligonucleotide
aaactgcagctaatattgatgagggttggc

SEQ ID NO: 15 - protein, 1102 aa, Arachis hypogaea
TATATACGGTCAAAAAAACGTATTATAAGAAGTATTACGAATATGATAAAAAAGATTATGACTGTGATTACGACAAAAAATATGATGACTATGATAAAAAATATTATGATCACGATAAAAAAGACTATGATTATGTTGTAGAGTATAAAAAGCATAAAAAACACTACCGTCTAGACACACCTCAAAATATTACTGATTTGTGTGCAGAATACCACAACACACAAATACATACGCTAAATGATAAGATATTTTCGTATACAGAATCTCTAGCTGGAAAAAGAGAGATGGCTATCATTACTTTTAAGAATGGTGCAACTTTTCAAGTAGAAGTACCAGGTAGTCAACATATAGATTCACAAAAAAAAGCGATTGAAAGGATGAAGGATACCCTGAGGATTGCATATCTTACTGAAGCTAAAGTCGAAAAGTTATGTGTATGGAATAATAAAACGCCTCATGCGATTGCCGCAATTAGTATGGCAAATGTCGACATGGGCGTCTTCACTTTCGAGGATGAAATCACCTCCACCGTGCCTCCGGCCAAGCTTTACAATGCTATGAAGGATGCCGACTCCATCACCCCTAAGATTATTGATGACGTCAAGAGTGTTGAAATTGTTGAGGGAAACGGTGGTCCCGGAACCATCAAGAAACTCACCATTGTCGAGGATGGAGAAACCAAGTTTATCTTGCACAAGGTGGAGTCAATAGATGAGGCCAACTATGCATACAACTACAGCGTTGTTGGAGGAGTGGCTCTGCCTCCCACGGCGGAGAAGATAACATTTGAGACAAAGCTGGTTGAAGGACCCAACGGAGGATCCATTGGGAAGCTTACTCTCAAGTACCACACCAAAGGAGATGCAAAGCCAGATGAGGAAGAGTTGAAGAAGGGTAAGGCCAAGGGTGAAGGTCTCTTCAGGGCTATTGAGGGTTACGTTTTGGCCAACCCTACTCAATATTAGCTGCAGGCATGCAAGCTTTAATGCGGTAGTTTATCACAGTTAAATTGCTAACGCAGTCAGGCACCGTGTATGAAATCTAACAATGCGCTCATCGTCATCCTCGGCACCGTCACCCTGGATGCTGTAGGCATAGGCTT

SEQ ID NO: 16 - protein, 438 aa, Arachis hypogaea
ATGGCCAAGTCCACCATCCTGGTAGCTCTCCTTGCCCTCGTCCTGGTGGCACACGCCTCCGCAATGAGGCGCGAGAGGGGGAGACAGGGGGACTCATCAAGCTGCGAGAGGCAGGTAGACAGGGTTAACCTCAAGCCCTGCGAGCAGCACATAATGCAGAGGATCATGGGCGAGCAAGAGCAGTACGACTCCTACGATATTAGGAGTACTCGATCCTCCGACCAGCAACAGAGGTGCTGCGATGAGCTGAACGAGATGGAGAACACACAGAGATGCATGTGCGAGGCATTGCAGCAGATAATGGAGAACCAGTGCGATAGGTTGCAGGACAGGCAAATGGTGCAGCAGTTCAAGAGAGAGCTCATGAACTTGCCCCAACAGTGTAACTTTAGGGCACCACAGCGTTGCGATTTGGACGTGAGTGGCGGCAGATGCTAA

SEQ ID NO: 17 - DNA, 28 bp, Artificial Sequence
  Oligonucleotide
aaagtcgacatggccaagtccaccatcc

SEQ ID NO: 18 - DNA, 29 bp, Artificial Sequence
  Oligonucleotide
aaagcggccgcttagcatctgccgccact

SEQ ID NO: 19 - DNA, 30 bp, Artificial Sequence
  Oligonucleotide
cgggaattcacacctcaaaatattactgat

SEQ ID NO: 20 - DNA, 28 bp, Artificial Sequence
  Oligonucleotide
aaggtcgacatttgccatactaattgcg

SEQ ID NO: 21 - protein, 1869 aa, Arachis hypogaea
ATGACTGGTGGACAGCAAATGGGTCGGGATCCGAATTCGAGCTCCGTCGACACGCATGCCAAGTCATCACCTTACCAGAAGAAAACAGAGAACCCCTGCGCCCAGAGGTGCCTCCAGAGTTGTCAACAGGAACCGGATGACTTGAAGCAAAAGGCATGCGAGTCTCGCTGCACCAAGCTCGAGTATGATCCTCGTTGTGTCTATGATCCTCGAGGACACACTGGCACCACCAACCAACGTTCCCCTCCAGGGGAGCGGACACGTGGCCGCCAACCCGGAGACTACGATGATGACCGCCGTCAACCCCGAAGAGAGGAAGGAGGCCGATGGGGACCAGCTGGACCGAGGGAGCGTGAAAGAGAAGAAGACTGGAGACAACCAAGAGAAGATTGGAGGCGACCAAGTCATCAGCAGCCACGGAAAATAAGGCCCGAAGGAAGAGAAGGAGAACAAGAGTGGGGAACACCAGGTAGCCATGTGAGGGAAGAAACATCTCGGAACAACCCTTTCTACTTCCCGTCAAGGCGGTTTAGCACCCGCTACGGGAACCAAAACGGTAGGATCCGGGTCCTGCAGAGGTTTGACCAAAGGTCAAGGCAGTTTCAGAATCTCCAGAATCACCGTATTGTGCAGATCGAGGCCAAACCTAACACTCTTGTTCTTCCCAAGCACGCTGATGCTGATAACATCCTTGTTATCCAGCAAGGGCAAGCCACCGTGACCGTAGCAAATGGCAATAACAGAAAGAGCTTTAATCTTGACGAGGGCCATGCACTCAGAATCCCATCCGGTTTCATTTCCTACATCTTGAACCGCCATGACAACCAGAACCTCAGAGTAGCTAAAATCTCCATGCCCGTTAACACACCCGGCCAGTTTGAGGATTTCTTCCCGGCGAGCAGCCGAGACCAATCATCCTACTTGCAGGGCTTCAGCAGGAATACGTTGGAGGCCGCCTTCAATGCGGAATTCAATGAGATACGGAGGGTGCTGTTAGAAGAGAATGCAGGAGGTGAGCAAGAGGAGAGAGGGCAGAGGCGATGGAGTACTCGGAGTAGTGAGAACAATGAAGGAGTGATAGTCAAAGTGTCAAAGGAGCACGTTGAAGAACTTACTAAGCACGCTAAATCCGTCTCAAAGAAAGGCTCCGAAGAAGAGGGAGATATCACCAACCCAATCAACTTGAGAGAAGGCGAGCCCGATCTTTCTAACAACTTTGGGAAGTTATTTGAGGTGAAGCCAGACAAGAAGAACCCCCAGCTTCAGGACCTGGACATGATGCTCACCTGTGTAGAGATCAAAGAAGGAGCTTTGATGCTCCCACACTTCAACTCAAAGGCCATGGTTATCGTCGTCGTCAACAAAGGAACTGGAAACCTTGAACTCGTGGCTGTAAGAAAAGAGCAACAACAGAGGGGACGGCGGGAAGAAGAGGAGGACGAAGACGAAGAAGAGGAGGGAAGTAACAGAGAGGTGCGTAGGTACACAGCGAGGTTGAAGGAAGGCGATGTGTTCATCATGCCAGCAGCTCATCCAGTAGCCATCAACGCTTCCTCCGAACTCCATCTGCTTGGCTTCGGTATCAACGCTGAAAACAACCACAGAATCTTCCTTGCAGGTGATAAGGACAATGTGATAGACCAGATAGAGAAGCAAGCGAAGGATTTAGCATTCCCTGGGTCGGGTGAACAAGTTGAGAAGCTCATCAAAAACCAGAAGGAATCTCACTTTGTGAGTGCTCGTCCTCAATCTCAATCTCAATCTCCGTCGTCTCCTGAGAAAGAGTCTCCTGAGAAAGAGGATCAAGAGGAGGAAAACCAAGGAGGGAAGGGTCCACTCCTTTCAATTTTGAAGGCTTTTAACAAGCTT

SEQ ID NO: 22 - DNA, 24 bp, Artificial Sequence
  Oligonucleotide
aaagtcgacgccaagtcatcacct

SEQ ID NO: 23 - DNA, 23 bp, Artificial Sequence
  Oligonucleotide
aaagcggccgcttagccacgcct

SEQ ID NO: 24 - DNA, 25 bp, Artificial Sequence
  Oligonucleotide
cggtctagacacacctcaaaatatt

SEQ ID NO: 25 - DNA, 21 bp, Artificial Sequence
  Oligonucleotide
aaactgcagttagccacgcct

SEQ ID NO: 26 - protein, 1224 aa, Arachis hypogaea
CTTTCTATGATTTTAACTGTCCAAGCCGCAAAATCTACTCGCCGTATAATAAAGCGTAGTAAAAATAAAGGAGGAGTATATATGGGTTATTACAAAAAATACAAAGAAGAGTATTATACGGTCAAAAAAACGTATTATAAGAAGTATTACGAATATGATAAAAAAGATTATGACTGTGATTACGACAAAAAATATGATGACTATGATAAAAAATATTATGATCACGATAAAAAAGACTATGATTATGTTGTAGAGTATAAAAAGCATAAAAAACACTACCGTCTAGACACACCTCAAAATATTACTGATTTGTGTGCAGAATACCACAACACACAAATACATACGCTAAATGATAAGATATTTTCGTATACAGAATCTCTAGCTGGAAAAAGAGAGATGGCTATCATTACTTTTAAGAATGGTGCAACTTTTCAAGTAGAAGTACCAGGTAGTCAACATATAGATTCACAAAAAAAAGCGATTGAAAGGATGAAGGATACCCTGAGGATTGCATATCTTACTGAAGCTAAAGTCGAAAAGTTATGTGTATGGAATAATAAAACGCCTCATGCGATTGCCGCAATTAGTATGGCAAATGTCGACGCCAAGTCATCACCTTACCAGAAGAAAACACTCGAGTATGATCCTCGTTGTGTCTATGATGGGGAGCGGACACGTGGCCGCCAACCCGGACGTAGGTACACAGCGAGGTTGAAGGAAGGCGGAAACATCTTCAGCGGCTTCACGCCGGAGTTCCTGGAACAAGCCGTGACAGTGAGGGGAGGCCTCAGAATCTTGAGCCCAGATAGAAAGGATGAAGATGAATATGAATACGATGAAGAGGATAGAAGGCGTGGCTAACTGCAGGCATGCAAGCTTTAATGCGGTAGTTTATCACAGTTAAATTGCTAACGCAGTCAGGCACCGTGTATGAAATCTAACAATGCGCTCATCGTCATCCTCGGCACCGTCACCCTGGATGCTGTAGGCATAGGCTTGGTTATGCCGGTACTGCCGGGCCTATTTCACTTTTTGCATTCTACAAACTGCATAACTCATATGTAAATCGCTCCTTTTTAGGTGGCACAAATGTGAGGCATTTTCGCTCTTTCCGGCAACCACTTCCAAGTAAAGTATAACACACTATACTTTATATTCATAAAGTGTGTGCTCTGCGAGGCTGTCGGCAGTGCCGACCAAAACCATAAAACCTTTAAGACCTTT

SEQ ID NO: 27 - protein, 1536 aa, Arachis hypogaea
TTCCGGCAGCAACCGGAGGAGAACGCGTGCCAGTTCCAGCGCCTCAATGCGCAGAGACCTGACAATCGCATTGAATCAGAGGGCGGTTACATTGAGACTTGGAACCCCAACAACCAGGAGTTCGAATGCGCCGGCGTCGCCCTCTCTCGCTTAGTCCTCCGCCGCAACGCCCTTCGTAGGCCTTTCTACTCCAATGCTCCCCAGGAGATCTTCATCCAGCAAGGAAGGGGATACTTTGGGTTGATATTCCCTGGTTGTCCTAGCACATATGAAGAGCCTGCACAACAAGGACGTCGATCTCAGTCCCAAAGACCACCAATACGTCTTCAAGGAGAAGACCAAAGCCAACAGCAACGAGATAGTCACCAGAAGGTGCACCGTTTCGATGAGGGTGATCTCATTGCAGTTCCCACCGGTGTTGCTTTCTGGCTCTACAACGACCACGACACTGATGTTGTTGCTGTTTCTCTTACTGACACCAACAACAACGACAACCAGCTTGATCAGTTCCCCAGGAGATTCAATTTGGCTGGGAACCACGAGCAAGAGTTCTTAAGGTACCAGCAACAAAGCAGACAAAGCAGACGAAGAAGCTTACCATATAGCCCATACAGCCCGCAAAGTCAGCCTAGACAAGAAGAGCGTGAATTTAGCCCTCGAGGACAGCACAGCCGCAGAGAACGAGCAGGACAAGAAGAAGAAAACGAAGGTGGAAACATCTTCAGCGGCTTCACGCCGGAGTTCCTGGAACAAGCCTTCCAGGTTGACGACAGACAGATAGTGCAAAACCTAAGAGGCGAGAACGAGAGTGAAGAAGAGGGAGCCATTGTGACAGTGAGGGGAGGCCTCAGAATCTTGAGCCCAGATAGAAAGAGACGTGCCGACGAAGAAGAGGAATACGATGAAGATGAATATGAATACGATGAAGAGGATAGAAGGCGTGGCAGGGGAAGCAGAGGCAGGGGGAATGGTATTGAAGAGACGATCTGCACCGCAAGTGTTAAAAAGAACATTGGTAGAAACAGATCCCCTGACATCTACAACCCTCAAGCTGGTTCACTCAAAACTGCCAACGATCTCAACCTTCTAATACTTAGGTGGCTTGGACTTAGTGCTGAATATGGAAATCTCTACAGGAATGCATTGTTTGTCCCTCACTACAACACCAACGCACACAGCATCATATATGCATTGAGGGGACGGGCTCACGTGCAAGTCGTGGACAGCAACGGCAACAGAGTGTACGACGAGGAGCTTCAAGAGGGTCACGTGCTTGTGGTGCCACAGAACTTCGCCGTCGCTGGAAAGTCCCAGAGCGAGAACTTCGAATACGTGGCATTCAAGACAGACTCAAGGCCCAGCATAGCCAACCTCGCCGGTGAAAACTCCGTCATAGATAACCTGCCGGAGGAGGTGGTTGCAAATTCATATGGCCTCCCAAGGGAGCAGGCAAGGCAGCTTAAGAACAACAACCCCTTCAAGTTCTTCGTTCCACCGTCTCAGCAGTCTCCGAGGGCTGTGGCTGTCGACAAGCTT

SEQ ID NO: 28 - protein, 10 aa, Arachis hypogaea
AKSSPYQKKT

SEQ ID NO: 29 - DNA, 30 bp, Artificial Sequence
  Oligonucleotide
gccaagtcatcaccttaccagaagaaaaca

SEQ ID NO: 30 - protein, 13 aa, Artificial Sequence
  Xaa stands for amino acid selenocysteine
  misc_feature (8)..(8): Xaa can be any naturally occurring amino acid
LEYDPRLXAAYDP

SEQ ID NO: 31 - DNA, 30 bp, Artificial Sequence
  Oligonucleotide
ctcgagtatgatcctcgttgtgtctatgat

SEQ ID NO: 32 - protein, 10 aa, Arachis hypogaea
GERTRGRQPG

SEQ ID NO: 33 - DNA, 30 bp, Artificial Sequence
  Oligonucleotide
ggggagcggacacgtggccgccaacccgga

SEQ ID NO: 34 - protein, 10 aa, Arachis hypogaea
RRYTARLKEG

SEQ ID NO: 35 - DNA, 30 bp, Artificial Sequence
  Oligonucleotide
cgtaggtacacagcgaggttgaaggaaggc

SEQ ID NO: 36 - protein, 15 aa, Arachis hypogaea
GNIFSGFTPEFLEQA

SEQ ID NO: 37 - DNA, 45 bp, Artificial Sequence
  Oligonucleotide
ggaaacatcttcagcggcttcacgccggagttcctggaacaagcc

SEQ ID NO: 38 - protein, 15 aa, Arachis hypogaea
VTVRGGLRILSPDRK

SEQ ID NO: 39 - DNA, 45 bp, Artificial Sequence
  Oligonucleotide
gtgacagtgaggggaggcctcagaatcttgagcccagatagaaag

SEQ ID NO: 40 - protein, 13 aa, Arachis hypogaea
DEDEYEYDEEDRG

SEQ ID NO: 41 - DNA, 45 bp, Artificial Sequence
  Oligonucleotide
gatgaagatgaatatgaatacgatgaagaggatagaaggcgtggc

SEQ ID NO: 42 - protein, 519 aa, Arachis hypogaea
ATGGCCAAGCTCACCATACTAGTAGCCCTCGCCCTTTTCCTCCTCGCTGCCCACGCATCTGCGAGGCAGCAGTGGGAACTCCAAGGAGACAGAAGATGCCAGAGCCAGCTCGAGAGGGCGAACCTGAGGCCCTGCGAGCAACATCTCATGCAGAAAATCCAACGTGACGAGGATTCATATGGACGGGACCCGTACAGCCCTAGTCAGGATCCGTACAGCCCTAGTCAGGACCCGGACAGACGTGATCCGTACAGCCCTAGTCCATATGATCGGAGAGGCGCTGGATCTTCTCAGCACCAAGAGAGGTGTTGCAATGAGCTGAACGAGTTTGAGAACAACCAAAGGTGCATGTGCGAGGCATTGCAACAGATAATGGAGAACCAGAGCGATAGGTTGCAGGGGAGGCAACAGGAGCAACAGTTCAAGAGGGAGCTCAGGAACTTGCCTCAACAGTGCGGCCTCAGGGCACCACAGCGTTGCGACTTGGAAGTCGAAAGTGGCGGCAGAGACAGATACTAA

SEQ ID NO: 43 - protein, 10 aa, Arachis hypogaea
QEPDDLKQKA

SEQ ID NO: 44 - protein, 10 aa, Arachis hypogaea
PGDYDDDRRQ

SEQ ID NO: 45 - protein, 10 aa, Arachis hypogaea
PRREEGGRWG

SEQ ID NO: 46 - protein, 10 aa, Arachis hypogaea
REREEDWRQP

SEQ ID NO: 47 - protein, 10 aa, Arachis hypogaea
EDWRRPSHQQ

SEQ ID NO: 48 - protein, 10 aa, Arachis hypogaea
QPKKIRPEGR

SEQ ID NO: 49 - protein, 10 aa, Arachis hypogaea
TPGQFEDFFP

SEQ ID NO: 50 - protein, 10 aa, Arachis hypogaea
SYLQEFSRNT

SEQ ID NO: 51 - protein, 10 aa, Arachis hypogaea
FNAEFNEIRR

SEQ ID NO: 52 - protein, 10 aa, Arachis hypogaea
EQEERGQRRW

SEQ ID NO: 53 - protein, 10 aa, Arachis hypogaea
DITNPINLRE

SEQ ID NO: 54 - protein, 10 aa, Arachis hypogaea
NNFGKLFEVK

SEQ ID NO: 55 - protein, 10 aa, Arachis hypogaea
GTGNLELVAV

SEQ ID NO: 56 - protein, 10 aa, Arachis hypogaea
ELHLLGFGIN

SEQ ID NO: 57 - protein, 10 aa, Arachis hypogaea
HRIFLAGDKD

SEQ ID NO: 58 - protein, 14 aa, Artificial Sequence
  Xaa stands for pyrrolysine
  misc_feature (3)..(3): Xaa can be any naturally occurring amino acid
  misc_feature (9)..(9): Xaa can be any naturally occurring amino acid
IDXAAIEKXAAAKD

SEQ ID NO: 59 - protein, 10 aa, Arachis hypogaea
KDLAFPGSGE

SEQ ID NO: 60 - protein, 10 aa, Arachis hypogaea
KESHFVSARP

SEQ ID NO: 61 - protein, 10 aa, Arachis hypogaea
PEKESPEKED

SEQ ID NO: 62 - protein, 10 aa, Arachis hypogaea
HASARQQWEL

SEQ ID NO: 63 - protein, 10 aa, Arachis hypogaea
QWELQGDRRC

SEQ ID NO: 64 - protein, 10 aa, Arachis hypogaea
DRRCQSQLER

SEQ ID NO: 65 - protein, 10 aa, Arachis hypogaea
LRPCEQHLMQ

SEQ ID NO: 66 - protein, 10 aa, Arachis hypogaea
KIQRDEDSYE

SEQ ID NO: 67 - protein, 10 aa, Arachis hypogaea
YERDPYSPSQ

SEQ ID NO: 68 - protein, 10 aa, Arachis hypogaea
SQDPYSPSPY

SEQ ID NO: 69 - protein, 10 aa, Arachis hypogaea
DRLQGRQQEQ

SEQ ID NO: 70 - protein, 10 aa, Arachis hypogaea
KRELRNLPQQ

SEQ ID NO: 71 - protein, 10 aa, Arachis hypogaea
QRCDLDVESG

SEQ ID NO: 72 - protein, 15 aa, Arachis hypogaea
IETWNPNNQEFECAG

SEQ ID NO: 73 - protein, 14 aa, Arachis hypogaea
MRRERGRGGDSSSS

SEQ ID NO: 74 - protein, 11 aa, Arachis hypogaea
KPCEQHIMQRI

SEQ ID NO: 75 - protein, 7 aa, Arachis hypogaea
YDSYDIR

SEQ ID NO: 76 - protein, 12 aa, Arachis hypogaea
CDELNEMENTQR

SEQ ID NO: 77 - protein, 10 aa, Arachis hypogaea
KRELRMLPQQ

SEQ ID NO: 78 - protein, 13 aa, Arachis hypogaea
CNFRAPQRCDLDV

SEQ ID NO: 79 - protein, 11 aa, Arachis hypogaea
DEITSTVPPAK

SEQ ID NO: 80 - protein, 9 aa, Arachis hypogaea
KDADSITPK

SEQ ID NO: 81 - protein, 12 aa, Arachis hypogaea
VEGNGGPGTIKK

SEQ ID NO: 82 - protein, 15 aa, Arachis hypogaea
ETKLVEGPNGGSIGK

SEQ ID NO: 83 - protein, 4 aa, Arachis hypogaea
GNGG

SEQ ID NO: 84 - protein, 6 aa, Arachis hypogaea
VEGPNG

SEQ ID NO: 85 - protein, 12 aa, Arachis hypogaea
KGDAKPDEEELK

SEQ ID NO: 86 - DNA, 29 bp, Artificial Sequence
  Oligonucleotide
cggtctagacacacctcaaaatattactg

SEQ ID NO: 87 - DNA, 27 bp, Artificial Sequence
  Oligonucleotide
aatctgcagttagcatctgccgccact

SEQ ID NO: 88 - protein, 103 aa, Arachis hypogaea
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 89 - protein, 623 aa, Arachis hypogaea
MTGGQQMGRDPNSSSVDTHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFNKL

SEQ ID NO: 90 - protein, 172 aa, Arachis hypogaea
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 91 - protein, 512 aa, Arachis hypogaea
FRQQPEENACQFQRLNAQRPDNRIESEGGYIETWNPNNQEFECAGVALSRLVLRRNALRRPFYSNAPQEIFIQQGRGYFGLIFPGCPSTYEEPAQQGRRSQSQRPPIRLQGEDQSQQQRDSHQKVHRFDEGDLIAVPTGVAFWLYNDHDTDVVAVSLTDTNNNDNQLDQFPRRFNLAGNHEQEFLRYQQQSRQSRRRSLPYSPYSPQSQPRQEEREFSPRGQHSRRERAGQEEENEGGNIFSGFTPEFLEQAFQVDDRQIVQNLRGENESEEEGAIVTVRGGLRILSPDRKRRADEEEEYDEDEYEYDEEDRRRGRGSRGRGNGIEETICTASVKKNIGRNRSPDIYNPQAGSLKTANDLNLLILRWLGLSAEYGNLYRNALFVPHYNTNAHSIIYALRGRAHVQVVDSNGNRVYDEELQEGHVLVVPQNFAVAGKSQSENFEYVAFKTDSRPSIANLAGENSVIDNLPEEVVANSYGLPREQARQLKNNNPFKFFVPPSQQSPRAVAVDKL

SEQ ID NO: 92 - protein, 145 aa, Arachis hypogaea
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 93 - protein, 157 aa, Arachis hypogaea
MGVFTFEDEITSTVPPAKLYNAMKDADSITPKIIDDVKSVEIVEGNGGPGTIKKLTIVEDGETKFILHKVESIDEANYAYNYSVVGGVALPPTAEKITFETKLVEGPNGGSIGKLTLKYHTKGDAKPDEEELKKGKAKGEGLFRAIEGYVLANPTQY

(19) United States
(12) Patent Application Publication
(10) Pub. No.: US 2018/0044384 A1
(43) Pub. Date: Feb. 15, 2018

(54) METHODS AND COMPOSITIONS TO REDUCE PEANUT-INDUCED ANAPHYLAXIS

(71) Applicants: Xiu-Min LI, Mamaroneck, NY (US); Zhenwen Zhou, Guangdong (CN); Zhigang LIU, Guangdong (CN); Ying SONG, Rego Park, NY (US)

(72) Inventors: Xiu-Min LI, Mamaroneck, NY (US); Zhenwen Zhou, Guangdong (CN); Zhigang LIU, Guangdong (CN); Ying SONG, Rego Park, NY (US)

(21) Appl. No.: 15/436,204

(22) Filed: Feb. 17, 2017

Related U.S. Application Data
(60) Provisional application No. 62/296,875, filed on Feb. 18, 2016.

(51) Int. Cl. C07K 14/28; C12N 15/70; C12R 1/63; C12N 1/20

(52) U.S. Cl. CPC C07K 14/28; C12N 1/20; C12N 15/70; C12R 1/63

(57) ABSTRACT

The present disclosure provides compositions comprising recombinant bacterial spores. The present disclosure is also directed to vaccine based compositions, which include recombinant bacterial spores that express CTB and a peanut protein(s) on their surfaces. This disclosure also provides methods for administering these compositions as a treatment or prevention of peanut allergy.

### CROSS REFERENCE TO RELATED APPLICATION

This application claims the benefit of priority from U.S. Provisional Application No. 62/296,875, filed Feb. 18, 2016, the entire contents of which are incorporated herein by reference.

### INCORPORATION BY REFERENCE OF SEQUENCE LISTING

The Sequence Listing in an ASCII text file, 33129_Seq_ST25.txt of 78 KB, created on Feb. 17, 2017, and submitted to the United States Patent and Trademark Office via EFS-Web, is incorporated herein by reference.

### BACKGROUND

Food allergy is a common disease, affecting up to 8% of children and 4% of adults in western countries, and is a major cause of anaphylaxis. Among the food allergies, peanut allergy has attracted great public attention because of its prevalence, severity of reactions, and frequent life-long persistence. Ingestion of small quantities of the allergen can lead to severe and potentially life-threatening reactions in patients. Avoidance of the allergen can prevent reactions, but because peanut is widely used in the food industry, patients with the allergy are at risk of consuming food products that are unintentionally cross-contaminated during the manufacturing procedure. This makes total avoidance of food allergens difficult to achieve. Therefore, for patients who are at risk for anaphylaxis, safe and affordable therapeutic approaches are needed.

Eleven peanut allergens have been described to date, being recognized by the WHO/IUIS and classified into different families and superfamilies of proteins (Saiz et al., Crit Rev Food Sci Nutr, (2013), 53, 722-737). Of these, Ara h1, Ara h2, and Ara h3 elicit the majority of specific immunoglobulin E (IgE) antibodies in allergic individuals. Ara h2 is a 16.7- to 18-kDa glycoprotein, initially found in crude peanut extracts and considered to be the most important peanut allergen due to the fact that more than 90% of sera IgE from peanut-sensitive patients recognize this allergen.

Oral immunotherapy (OIT) has emerged as the most actively investigated therapy for peanut allergy. In OIT protocols, allergic patients are desensitized to the allergic food, which protects them against reactions from accidental ingestions, but adverse reactions during upon dosage are reported frequently. In a recently large peanut OIT study, ninety-three percent of subjects experienced some symptoms, mostly upper respiratory and abdominal distress (Hofmann et al., J Allergy Clin Immunol, (2009), 124, 286-291). Safety is of the paramount importance during such trials.

The Vibrio cholerae derived Cholera Toxin B (CTB), is non-toxic and is an important component of an oral cholera vaccine proven to be safe, even for pregnant women, which elicits long lasting protective immunity (Hashim et al., Plos Negl Trop Dis, (2012), 6, e1743. doi: 10.1371/journal.pntd.0001743). CTB when mucosally co-administered with antigens can induce antigen-specific tolerance in animal models and humans (Basset et al., Toxins (Basel) (2010), 2, 1774-1795; Sun et al., Scand J Immunol (2010), 71, 1-11). This makes the use of CTB a potentially important strategy to treat allergic disorders.

Bacillus subtilis (B. subtilis) is a spore-forming, Gram-positive bacterium used for industrial enzyme production. It is regarded as a nonpathogen and has been widely used as a probiotic for both humans and animals consumptions. For its safety and stability, B. subtilis spore has been used recently as an attractive delivery vehicle to extreme acidic gastrointestinal tract (Valdez et al., J Appl Microbiol, (2014), 117, 347-357; Wang et al., Vaccine, (2014), 32, 1338-1345).

### BRIEF DESCRIPTION OF THE DRAWINGS

FIG. 1. Schematic representation of genetic engineering. Synthesized Cholera Toxin B (CTB) DNA was cloned to plasmid pET24-Ara h2 and transformed to E. coli BL21, followed by CTB-Ara h2 DNA subcloned to plasmid pus186-CotC and finally the recombinant pus186-CotC-CTB-Ara h2 plasmid was transformed to B. subtilis WB600.

FIG. 2. Mice experimental design. C3H/HeJ mice were administrated with peanut orally by intragastric lavage weekly for sensitization from week 0 to week 5, and boosted at week 6, 8 and 15. Peanut-allergic mice were treated with recombinant spores expressing CTB-Ara h2 for 3 consecutive days weekly from week 9 to week 14. Mice were challenged at week 19.

FIGS. 3A-3B. SDS-PAGE and Western blot analysis of proteins extracted from spores. Sporulation was induced in DSM medium by exhaustion method as described in Zhou et al. (Vaccine. 2008 Mar. 28; 26(15):1817-25), coat proteins of spores were extracted in SDS-DTT buffer by sonication. (A) Coomassie blue stained 12% SDS-PAGE of proteins extracted from recombinant spores and CotC spores. Lane 1: protein molecular weight markers (kDa); Lane 2: CotC-CTB-Ara h2 expressing strain, arrow points to fusion protein; Lane 3: CotC strain. (B) Western blot analysis of proteins extracted from recombinant spores and CotC spores using Ara h2-specific antiserum. Lane 1: CotC-CTB-Ara h2 expressing strain, arrow points to fusion protein; lane 2: CotC strain.

FIGS. 4A-4B. Anaphylaxis scores and temperatures of mice 30 minutes after peanut challenge. (A) Anaphylactic symptoms scores. (B) Core body temperatures. Each dot represents an individual mouse. Horizontal bar indicates the mean. *p<0.05 vs sham.

FIG. 5. Plasma histamine levels after peanut challenge. Blood was collected 30 minutes after peanut challenge and individual samples from groups were tested by ELISA for plasma histamine. Horizontal bar indicates the mean. *p<0.05 vs sham.

FIGS. 6A-6D. Effect of Peanut (PN)+Cholera Toxin B (CTB) oral vaccine on PN specific (s)-IgE (sIgE) and anaphylaxis in mice with established peanut allergy (PA). C3H/HeJ female mice were sensitized with 10 mg PN plus 20 μg of CT for 5 weeks at weekly interval and boosted with 50 mg of PN and 20 μg CT at week 6 and again week 8 at which time PN hypersensitivity was established as reported by Qu et al (named PA mice). PN (2.5 mg equivalent protein)+CTB (20 μg) treatment began wk 8 weekly for 6 weeks. PN or CTB alone or water (Sham) treated PA mice and Naïve mice were controls. Mice were challenged at week 14. (A). Scheme of experimental protocol. (B). PN sIgE at wk14 one day after treatment. (C). Anaphylactic scores ranging from 0 no reaction to 5 death and (D) core body temperatures were assessed 30 min following PN oral challenge. *p<0.05, **, p<0.01. vs sham (N=4-5).

FIG. 7A-7H. Effect of PN+CTB vaccination during gestation and lactation on mothers PA. PN-sensitized female mice were fed with water (sham), or PN+CTB or CTB alone or PN alone for 6 weeks during gestation and lactation. One week later (after weaning), blood was collected and mice were challenged. (A) Serum PN specific IgE levels. (B-D): Core body temperatures, anaphylactic scores and plasma histamine levels. (E-F): Serum PN sIgG2a and Fecal PN sIgA levels. (G-H) and H IL-4 and IL-10 cytokine levels in splenocytes (SPC) and Mesenteric lymph node (MLN) cell cultures. N=8-11. *p<0.05, **p<0.01 vs. sham

FIG. 8A-8H. Offspring response to PN sensitization and challenge. Offspring of sham fed mothers (Sham), PN+CTB fed mothers (PN+CTB), CTB fed mother (CTB) and PN fed mothers were i.g. sensitized and challenged as in FIG. 6. Naïve mice were used as normal controls. PN-specific IgE in sera (A) and PN-specific IgA in feces (B) one day prior to challenge. (C-E): Anaphylactic scores, body temperature and plasma histamine levels of offspring following PN oral challenge. (F-G): IL-4 and IL-10 cytokine levels in offspring MLN cell cultures. (H): CD4⁺CD25⁺T regulatory cells vs. CD4+ T cells in SPCs analyzed by flow cytometry. N=8-11 over 2 batches. *p<0.05 vs sham.

FIG. 9A-9B. Treatment with mixed spore constructs significantly increases the T_{reg} cell population in splenocytes cultured with Crude Peanut Extract (CPE). Splenocytes isolated from mixed spores-treated, sham treated PA mice, and naïve mice were cultured with CPE. After 3 days of culture, splenocytes were labeled with fluorescent anti CD3, CD4, CD25 and Foxp3 antibodies. Data were analyzed by FlowJo software. (A) A representative plot of CD4⁺CD25⁺Foxp3⁺ T_{reg}. (B) Percent of CD4⁺CD25⁺Foxp3⁺ T_{reg} in CD4⁺ T cells in splenocyte cultures in response to CPE. N=3, p<0.05 vs. sham.

FIG. 10. Schematic representation of genetic engineering. Synthesized CTB DNA was cloned to plasmid pET24-Ara h2 and transformed to E. coli BL21, followed by CTB-Ara h2 DNA subcloning to plasmid Pus186-CotC. Finally, the recombinant Pus186-CotC-CTB-Ara h2 plasmid was transformed to Bacillus subtilis WB600. Method of Construction of gene fusion: CTB DNA was amplified by PCR using the synthesized CTB DNA as template and the following designed primers. The designed primers include: forward primer: 5′CGGGCTAGCACACCTCAAAATATTACTGAT3′ with a NheI site (SEQ ID NO: 1), reverse primer: 5′GGCGTCGACATTTGCCATACTAATTGCG3′ with a SalI site (SEQ ID NO: 5). The PCR conditions were as follows: 94° C. for 4 m followed by 35 cycles of 94° C. for 30 s, 55° C. for 30 s and 72° C. 60 s, and the reaction continued for 10 min at 72° C. after the last cycle. The purified PCR product was digested with NheI, SalI and cloned into NheI/SalI double digested pET 24-Arah2 plasmid. CTB-Arah2 DNA was amplified by using the constructed pET24-CTB-Arah2 plasmid as template. The PCR primers included: forward primer: 5′CGGTCTAGAGACACCTCAAAATATTACTGATT 3′ with XbaI site (SEQ ID NO: 3), reverse primer: GGCAAGCTTTTAAAGCTTGTTAAAAGCCTT with HindIII (SEQ ID NO: 6). The purified PCR product was double digested by XbalI/HindIII and ligated to the 3′ end of the CotC gene in pUS186-CotC plasmid constructed and transformed into B. subtilis WB600. The sequences of the fusion gene were confirmed by sequence analysis.

FIG. 11A-11D. BCAV protects female C3H/HeJ mice from PA anaphylaxis and induced a beneficial immune response. Mice received epicutaneous sensitization with PN (1 mg)+CT(10 μg). BCAV treatment (1×10⁹ spores) or vehicle/sham began 6 weeks after the initial sensitization) i.g 3 times (Mon. Tues. and Weds.) per week at weekly intervals for 4 weeks. 3 weeks post therapy, blood was collected, mice were challenged, splenocytes were cultured and cytokine levels were determination. (A). PN-sIgE, (B). Core body temperatures, (C) Splenocyte culture IL-10 levels, and (D) Splenocyte culture IL-4 levels. *p<0.05 vs sham.

FIG. 12A-12C. Offspring of ARM showed increased susceptibility to PA. Female BALB/c mice with Rag Weed (RW)-induced AR were bred with naïve males. Offspring of O-ARM and O-NM were i.g. sensitized with suboptimal dose of peanut (5 mg)+CT for 3 weeks and then i.g. challenged with 200 mg PN/mouse. Blood was collected from offspring one day before challenge and PN sIgE levels were determined by ELISA (A). Symptom scores (B) and core body temperatures (C) were measured 30 minutes after challenge (N=5-6). *p<0.05, ***, p<0.001, vs. ONM; #p<0.05, vs. Naïve.

FIG. 13A-13B. CTB+PN vaccine altered DNA methylation status at IL4 and Foxp3 promoters. PN-sensitized female mice were fed water (sham), CTB+PN, CTB, or PN for 6 weeks during gestation and lactation. Blood samples were collected before challenge and purified genomic DNA from peripheral blood leucocytes (PBL) underwent bisulfite conversion, PCR amplification, and pyrosequencing. DNA methylation at CpG-71 CpG-53 and CpG-50 sites of the Foxp3 promoters (A) and CpG-408 and CpG-393 sites of the IL-4 promoter (B) in Sham, CTB+PN, CTB alone and PN alone fed mice. *p<0.05; **p<0.01 vs. sham. N=4-5.

FIG. 14A-14B. DNA methylation status of IL-4 (A) and Foxp3 (B) promoters in offspring CD4+T cells. Offspring from sham, PN+CTB, CTB alone and PN alone fed PAM were PN sensitized and challenged, and naive mice were included as controls. Offspring splenocytes were isolated from each group of 8-11 mice over 2 batches. Splenocyte CD4+ T cells were isolated using Mouse CD4+ T Cell Isolation Kit. Genomic DNA was purified from CD4⁺ cells and underwent bisulfate treatment, PCR amplification, and pyrosequencing. *p<0.05; **p<0.01 vs. sham. N=5.

FIG. 15. microRNA levels in fetal spleens from sham treated and mixed spore constructs treated PA mice. miR-106a and miR-98 levels were evaluated in triplicate using two-step TaqMan MicroRNA assays. The 2-ΔCT method was used for quantification. Values are fold changes in splenocytes from mixed spores treated PNA mice compared to splenocytes from sham treated PNA mice with normalization to endogenously expressed small RNAs (U6 snRNA) control. N=3-4/group. *, p<0.05.

FIG. 16A-16B. Increased DNA methylation of IL-4 promoter (A) and decreased DNA methylation at Foxp3 promoter (B) in oocytes of BCAV treated PA-M. Oocyte retrieval method: BCAV treated PAM were superovulated by intraperitoneal injection of 5 IU of pregnant mare's serum followed by 5 IU of human chorionic gonadotropin 48 hrs later. Mice were euthanized the following morning and the oviducts were placed in a 35-mm tissue culture dish containing FHM media at RT. Individual oviducts were sequentially transferred to a 35 mm tissue culture dish containing FHM media with hyaluronidase prewarmed to 37° C., and observed under a dissecting microscope. Each oviduct was immobilized behind the ampulla with forceps and the outer wall of the ampulla was opened by tearing to release the cumulus mass. To prevent contamination of oocyte sampled by cumulus cells, complete separation of oocytes from cumulus cells were performed by removing zona pellucida by incubation in warm acidic Tyrode's solution. Individual oocytes were recovered using a custom prepared holding pipette controlled with Narashige coarse and fine manipulators and washed through three changes of FHM media. Oocytes pooled from 3 mice (expected yield of 10-15 oocytes/mouse) were placed RLT buffer. *p<0.05; **p<0.01 vs. sham. N=3 sets from 9 mice/group.

FIG. 17A-17G. Reduction of allergic reactions and peanut specific and Ara h2 specific IgE response in peanut allergic mice by BCA2 vaccine. (A) Protocol: Orally sensitized peanut allergic female C3H/HeJ mice received BCAV (1×10⁹) or BS at the same number of spores or PN at equivalent dose of Ara h2 to BCAV daily beginning at 9 weeks for 5 weeks. Four weeks post therapy, mice were challenged i.g. with PN (200 mg) and anaphylactic reactions were evaluated 30 min later. BCAV treated mice showed significantly lower anaphylactic symptom scores (B) higher core body temperatures (C) lower plasma histamine levels (D) lower PN-IgE (E) and Arah2-IgE (F) than sham treated mice. *p<0.05 vs sham. N=5/group. BCAV (BCA2) stands for “Bacillus subtilis spores surface expressing CTB fused to Ara h2 vaccine”; BS stands for “Bacillus subtilis spores contains the mock vector without Ara h2/CTB”; PN stands for “peanut”; i.g. stands for “intragastrical gavage”.

FIG. 18A-18H. Maternal BCA2 vaccine prevents PA development and induction of tolerogenic immunity in high risk offspring. Female Peanut Allergic Mice (PAM) generated as in FIG. 17 received BCAV (1×10⁹) or BS at the same number of spores or PN at equivalent dose of Ara h2 to BCAV i.g. 3 times per week for 4 weeks. One week following treatment, mice were mated with native males and there was no peanut exposure during pregnancy and lactation. F1 offspring at 8-12 days old received e.c. sensitization with PN+CT 3 times weekly and followed i.g. PN sensitization weekly twice. 4 weeks later, they were i.g. challenged with PN (200 mg). (A) and (B). Serum PN-, Arah2-specific IgE levels. (C) Body temperature. (D) Plasma histamine. (E) Fecal PN-specific IgA. F-G. Splenocyte IL-10 and IL-4 levels. (H) Percent of SPC T_{reg}s among CD4+ T cells. *p<0.05 vs sham (n=3-5/group).

FIG. 19A-19D. PCR identification of Arah8, CTB, CTB-Ara h8 clone in pET28a and recombinant Pus186cotC-CTB-Arah8 plasmid. (A): PCR of Ara h8. Lane1-2: PCR production of Arah8 using synthesized Arah8 sequences as template; Lane3: DNA marker DL10000. (B): PCR of CTB. Lane1-2: PCR production of CTB using constructed pET28a-CTB-Ara h2 as template; Lane3: DNA marker DL1000. (C): CTB-Ara h8 clone in pET28a. Lane1-2: pET-28a CTB-Arah8 plasmid; Lane3: DNA marker DL10000; Lane4: CTB PCR production using pET-28a CTB-Arah8 plasmid as template; Lane5: pET28a-Arah8 double enzyme by EcoRI, SalI. (D): PCR identification of recombinant Pus186cotC-CTB-Arah8 plasmid. Lane1: Pus186cotC-CTB-Arah8 plasmid; Lane2: DNA Marker DL10000; Lane3: CTB-Arah8 PCR production using Pus186cotC-CTB-Arah8 plasmid as template.

FIG. 20A-20C. PCR identification of CTB, recombinant pET28-CTB-Arah6 and recombinant CTB-Ara6 in Pus186cotC-CTB-Ara h6 plasmid. (A): PCR of CTB. Lane1: DNA Marker DL1000; Lane2-4: CTB PCR product. (B): PCR identification of recombinant pET28-CTB-Ara h6. Lane1:Arah6 PCR product using recombinant pET28-CTB-Ara h6 plasmid as template; Lane2:pET28a double enzyme by Sal1,Not1; Lane3:DNA Marker DL10000. (C): PCR identification of recombinant CTB-Ara6 in Pus186cotC-CTB-Arah6 plasmid. Lane 1: CTB PCR product; Lane3-4: CTB PCR product using recombinant Pus186cotC-CTB-Ara h6 plasmid as template; Lane 7: DNA Marker DL1000; Lane8, 10-11: Ara h6 PCR production using recombinant Pus186cotC-CTB-Ara h6 plasmid as template.

FIG. 21A-21C. PCR identification of epitope h1&3, recombinant CTB-Epitope in pET28a-CTB-Epitope h1&3 and recombinant CTB-Epitope h1&3 in Pus186cotC-CTB-Epitope 1&3 plasmid. (A) Epitope 1&3 PCR Lane1:1000 marker; Lane2: Epitope 1&3 PCR production (258 bp) using synthesized epitope sequences as template; Lane3:10000 marker. (B) PCR identification of recombinant CTB-Epitope in pET28a-CTB-Epitope 1&3. Lane1:1000 marker. Lane2-6: PCR production of CTB-Epitope 1&3(567 bp) using constructed pET28a-CTB-Epitope 1&3 plasmid as template. (C) PCR identification of recombinant CTB-Epitope 1&3 in Pus186cotC-CTB-Epitope 1&3 plasmid; Lane1-4: PCR production of CTB-Epitope 1&3 (567 bp) using constructed Pus186cotC-CTB-Epitope 1&3 plasmid as template; Lane5: 10000 marker.

FIG. 22. Nucleotide sequence encoding the peanut antigen Ara h1 (SEQ ID NO 21). The nucleotides underlined represent some exemplary epitopes.

FIG. 23. Nucleotide sequence encoding the peanut antigen Ara h3 (SEQ ID NO 27). The nucleotides underlined represent some exemplary epitopes.

### DETAILED DESCRIPTION OF THE INVENTION

The present disclosure is directed to compositions containing recombinant bacterial spores expressing Cholera Toxin B (CTB) and one or more peanut antigens, and methods of using such compositions for inducing tolerance or reducing sensitivity to a peanut allergen or peanut allergy in a subject. The invention is predicated at least in part on the discovery by the present inventors that by utilizing bacterial spores (such as spores of B. subtitlis) as recombinant expression carriers for CTB and peanut antigens, effective and safe tolerance can be induced with a much lower amount of CTB and peanut antigens.

The term “subject” encompasses human or non-human animal such as a companion animal, livestock animal or captured wild animal. In some embodiments, the subject is a subject who has peanut allergy. In some embodiments, the subject is a pregnant woman. In some embodiments, the subject is an adult, and in other embodiments, the subject is a child.

The term “inducing tolerance” as used herein includes reducing sensitivity to an allergen or an allergen associated with an allergy. Hence, it encompasses reducing sensitivity to an allergy as well as reducing intolerance to an allergen-induced allergy.

The term “allergen” includes any substance which is capable of stimulating a typical hypersensitivity reaction (mainly through inducing an IgE response) in a subject. In specific embodiments of this disclosure, an allergen is a peanut allergen.

The term “antigen” means a substance that induces an immune response in the body, especially the production of antibodies.

The term “recombinant bacterial spore” refers to a spore of a bacterial cell that has been genetically engineered as described herein that express CTB and one or more peanut antigens.

CTB and Peanut Antigens

Cholera Toxin B (CTB) has been described in the art. See, e.g., Basset, C. et al. (2010), Toxins (Basel) 2, 1774-1795; Sun J B. Et al., (2010), Scand J Immunol, 71, 1-11. In some embodiments, CTB that is expressed by the recombinant bacterial spores of this invention includes an amino acid sequence that is substantially identical (i.e., at least 85%, 90%, 95%, 98%, 99% or greater) with the amino acid sequence as set forth in SEQ ID NO: 88.

Peanut antigens expressed by the recombinant bacterial spores of this invention can be selected from the group consisting of an Ara h1 antigen, an Ara h2 antigen, an Ara h3 antigen, an Ara h6 antigen, or an Ara h8 antigen. Antigens used in this context are meant to include an Ara h molecule in full or in part that comprises at least one (i.e., one or more) antigenic epitopes of the Ara h molecule. For example, an Ara h2 antigen include a full length or substantially full length Ara h2 molecule, or a molecule containing at least one antigenic epitope of full length Ara h2 molecule. By “antigenic epitope” is meant a peptide that is of sufficient length to induce an antigenic response in a recipient, e.g., at least 8, 9, 10, 11, 12, 13, 14, 15 amino acids or longer in length. In some embodiments, an antigenic epitope refers to a peptide that binds to IgE or induces an IgE response in a recipient.

In some embodiments, Ara h1 has an amino acid sequence that is substantially identical with the amino acid sequence as set forth in SEQ ID NO: 89. Exemplary epitopes of Ara h1 suitable for use herein are set listed in the table below (Table 1). These epitopes have been identified as Ara h1 IgE-binding epitopes (see, e.g., Burks et al. (1997), Eur. J. of Biochemistry, 245(2), 334-339).

In some embodiments, peanut antigens expressed by the recombinant bacterial spores of this invention include Ara h2 or one or more epitope(s) thereof. In some embodiments, peanut antigens expressed by the recombinant bacterial spores include a full length or substantially full length Ara h2 molecule. In some embodiments, Ara h2 has an amino acid sequence that is substantially identical with the amino acid sequence as set forth in SEQ ID NO: 90. Exemplary epitopes of Ara h2 suitable for use herein are set forth below in Table 2. These epitopes have been identified as Ara h2 IgE-binding epitopes in Stanley et al., (1997), Archives of Biochemistry & Biophysics, 342(2), 244.

TABLE 1
Ara h1 Epitopes

| Peptide | Amino acid sequence |
|---|---|
| 1 | AKSSPYQKKT (SEQ ID NO: 28) |
| 2 | QEPDDLKQKA (SEQ ID NO: 43) |
| 3 | LEYDPRLUYD (SEQ ID NO: 30) |
| 4 | GERTRGRQPG (SEQ ID NO: 32) |
| 5 | PGDYDDDRRQ (SEQ ID NO: 44) |
| 6 | PRREEGGRWG (SEQ ID NO: 45) |
| 7 | REREEDWRQP (SEQ ID NO: 46) |
| 8 | EDWRRPSHQQ (SEQ ID NO: 47) |
| 9 | QPKKIRPEGR (SEQ ID NO: 48) |
| 10 | TPGQFEDFFP (SEQ ID NO: 49) |
| 11 | SYLQEFSRNT (SEQ ID NO: 50) |
| 12 | FNAEFNEIRR (SEQ ID NO: 51) |
| 13 | EQEERGQRRW (SEQ ID NO: 52) |
| 14 | DITNPINLRE (SEQ ID NO: 53) |
| 15 | NNFGKLFEVK (SEQ ID NO: 54) |
| 16 | GTGNLELVAV (SEQ ID NO: 55) |
| 17 | RRYTARLKEG (SEQ ID NO: 34) |
| 18 | ELHLLGFGIN (SEQ ID NO: 56) |
| 19 | HRIFLAGDKD (SEQ ID NO: 57) |
| 20 | IDOIEKOAKD (SEQ ID NO: 58) |
| 21 | KDLAFPGSGE (SEQ ID NO: 59) |
| 22 | KESHFVSARP (SEQ ID NO: 60) |
| 23 | PEKESPEKED (SEQ ID NO: 61) |

TABLE 2
Ara h2 Epitopes, core binding amino acids are
bold and underlined

| Peptide | AA Sequence |
|---|---|
| 1 | HASARQQWEL (SEQ ID NO: 62) |
| 2 | QWELQGDRRC (SEQ ID NO: 63) |
| 3 | DRRCQSQLER (SEQ ID NO: 64) |
| 4 | LRPCEQHLMQ (SEQ ID NO: 65) |
| 5 | KIQRDEDSYE (SEQ ID NO: 66) |
| 6 | YERDPYSPSQ (SEQ ID NO: 67) |
| 7 | SQDPYSPSPY (SEQ ID NO: 68) |
| 8 | DRLQGRQQEQ (SEQ ID NO: 69) |
| 9 | KRELRNLPQQ (SEQ ID NO: 70) |
| 10 | QRCDLDVESG (SEQ ID NO: 71) |

In some embodiments, peanut antigens expressed by the recombinant bacterial spores of this invention include, in addition to Ara h2 or an epitope(s) thereof, also include Ara h3, Ara h6, or Ara h8, or an epitope or epitopes thereof.

In some embodiments, Ara h3 has an amino acid sequence that is substantially identical with the amino acid sequence as set forth in SEQ ID NO: 91. Exemplary epitopes of Ara h3 suitable for use herein are set forth below:

TABLE 3
Ara h3 Epitopes, These epitopes have also
been described in Rabjohn et al., (1999),
J. of Clin.l Investigation, 103(4), 535-45.

| Peptide | AA Sequence |
|---|---|
| 1 | IETWNPNNQEFECAG (SEQ ID NO: 72) |
| 2 | GNIFSGFTPEFLEQA (SEQ ID NO: 36) |
| 3 | VTVRGGLRILSPDRK (SEQ ID NO: 38) |
| 4 | DECEYEYDEEDRG (SEQ ID NO: 40) |

In some embodiments, Ara h6 has an amino acid sequence that is substantially identical with the amino acid sequence as set forth in SEQ ID NO: 92. Exemplary epitopes of Ara h6 suitable for use herein are set forth below:

TABLE 4
Ara h6 Epitopes

| Peptide | AA Sequence |
|---|---|
| 1 | MRRERGRGGDSSSS (SEQ ID NO: 73) |
| 2 | KPCEQHIMQRI (SEQ ID NO: 74) |
| 3 | YDSYDIR (SEQ ID NO: 75) |
| 4 | CDELNEMENTQR (SEQ ID NO: 76) |
| 5 | KRELRMLPQQ (SEQ ID NO: 77) |
| 6 | CNFRAPQRCDLDV (SEQ ID NO: 78) |

In some embodiments, Ara h8 has an amino acid sequence that is substantially identical with the amino acid sequence as set forth in SEQ ID NO: 93. Exemplary epitopes of Ara h8 suitable for use herein are set forth below:

TABLE 5
Ara h8 Epitopes

| Peptide | AA Sequence |
|---|---|
| 1 | DEITSTVPPAK (SEQ ID NO: 79) |
| 2 | KDADSITPK (SEQ ID NO: 80) |
| 3 | VEGNGGPGTIKK (SEQ ID NO: 81) |
| 4 | ETKLVEGPNGGSIGK (SEQ ID NO: 82) |
| 5 | GNGG (SEQ ID NO: 83) |
| 6 | VEGPNG (SEQ ID NO: 84) |
| 7 | KGDAKPDEEELK (SEQ ID NO: 85) |

In specific embodiments, peanut antigens expressed by the recombinant bacterial spores of this invention include Ara h2, in combination with at least one (i.e., one or more) epitope of Ara h1, Ara h3, Ara h6, or Ara h8. In particular embodiments, peanut antigens expressed by the recombinant bacterial spores of this invention include Ara h2, in combination with one or more epitopes from each of Ara h1, Ara h3, Ara h6, and Ara h8.

In some embodiments, recombinant bacteria can be generated such that CTB and a peanut antigen are expressed on the cell surface of different bacterial cells or spores, and the different bacterial cells or spores can be mixed to obtain a composition containing both CTB and a peanut antigen. In some embodiments, recombinant bacteria can be generated such that CTB and a peanut antigen are co-expressed on the cell surface of the same bacterial cells or spores, e.g., through expression based on a fusion protein, or through selecting bacterial cells transformed with both/separate expression vectors encoding CTB and a peanut antigen, respectively. Similarly, where multiple peanut antigens are expressed, a peanut antigen can be expressed on the cell surface of the same bacterial cells/spores that also express CTB and/or another peanut antigen, or on the cell surface of different bacterial cells/spores that express CTB and/or another peanut antigen, and the cells/spores expressing different antigens can be mixed together prior to administration.

Generation of Recombinant Bacteria and Spores

Nucleic acid molecules encoding CTB and/or one or more peanut antigens can be introduced into appropriate bacterial cells by using conventional transformation techniques.

In some embodiments, other DNA, e.g., DNA encoding cell adherence proteins, may be introduced into and expressed by bacteria in addition to CTB and/or peanut antigen-encoding DNA(s). The antigen and adherence proteins may be expressed as fusion proteins with endogenous bacterial cell wall or spore coat associated proteins, or any other desired proteins. By “adherence protein” is meant one which allows the cell in which it is expressed to adhere to another cell, preferably a vertebrate animal cell, more preferably a mammalian cell. Examples of such proteins are Invasin (Inv) from Yersinia enterocolitica or Colonization Factor Antigens (CFAs) from enterotoxigenic E. coli. Inv, CFAs or other adherence proteins may be both protective antigens and a mechanism to allow colonization of the vector strain in the intestinal tract. These proteins will generally be expressed so that they are at least partially exposed on the surface of the spore or vegetative bacterial cell to ensure that they have access to binding sites on animal cells.

Bacterial species capable of forming spores are suitable for use in this invention. In some embodiments, the bacterial cell which is capable of forming spores is probiotic. A probiotic microorganism is generally a live eukaryotic or a prokaryotic organism which has a beneficial property when given to a subject. In one aspect, a probiotic microorganism complements the existing microflora in the subject. Hence, a probiotic agent is a live microorganism which can confer a health benefit to a host subject. In the context of the present invention, a probiotic bacteria can be provided as a culture of the bacteria, which can be used in the administration directly, or provided in a dietary supplement, or may be freeze-dried and reconstituted prior to use.

Examples of probiotic bacteria include species of Lactobacillus, Escherichia, Bacillus, Bifidobacterium, Saccharomyces and Streptococcus. Specific examples of probiotic bacteria suitable for use in the present invention are listed in Table 6 (below).

Other genera are also suitable for use, including the genera Clostridium, Actinomycetes, Streptomyces, Nocardia, or any spore forming bacterium. Implementation of the invention in some bacteria (e.g., human pathogens like strains of E. coli and strains of Salmonella) may require the use of mutants which lack expression of toxins or other pathogenic characteristics.

In a specific embodiment, the bacteria used is a strain of Bacillus subtilis.

In some embodiments, bacterial spores are stored and/or provided as a dried composition in solid form (e.g., powder, granules, or a lyophilized form). In another embodiment, bacterial spores are stored and/or provided in a semi-solid or liquid composition.

In some embodiments, recombinant bacterial spores expressing CTB and one or more peanut antigens are used in the administration and are capable of germination following ingestion. Upon ingestion and germination, the same or a different peanut antigen (e.g., a shorter peptide) can be expressed on the surface of or secreted by the resulting vegetative bacteria. This embodiment has the advantage of exposing the animal to the desired antigen immediately upon ingestion, and continuing antigenic exposure through bacterial germination and vegetative cell growth.

In some embodiments, the genetically engineered spores may be treated prior to oral administration to initiate germination. This is also known as “activation” and can be achieved by aging or more preferably by heat treatment and exposure to germinants, e.g., applying heat shock and L-alanine or a mixture of glucose, fructose, asparagine, and KCl (GFAK). This activation allows spores to retain surface proteins, but makes them more permeable to specific germinants, allowing them to grow into vegetative cells more efficiently. A method of activating spores prior to oral administration is to suspend them in a hot broth or water, then cool the suspension to a suitable temperature prior to administration to the animal, e.g., a human.

TABLE 6
Examples of probiotic bacteria species that can be used. Specific
strains from these species are also described in Meijerink et al., (2012),
Fems Immunology & Medical Microbiology, 65(3), 488-496.

Species
B. animalis
B. lactis
B. lactis
B. longum
L. acidophilus
L. acidophilus
L. acidophilus
L. casei
L. casei
L. casei
L. casei
L. fermentum
L. gasseri
L. johnsonii
L. plantarum
L. plantarum
L. plantarum
L. plantarum
L. reuteri
L. reuteri
L. reuteri
L. rhamnosus
L. rhamnosus
L. rhamnosus
L. rhamnosus
L. rhamnosus
L. salivarius
L. salivarius

The bacterial spores or resultant vegetative cell of the invention preferably has a residence time in the digestive tract of the animal of at least one day, more preferably at least two to ten days, or possibly permanent colonization.

Therapeutic Compositions and Methods

The bacterial spores can be mixed with a pharmaceutically acceptable carrier prior to administration. For the purposes of this disclosure, “a pharmaceutically acceptable carrier” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, and the like. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

In specific embodiments, a pharmaceutically acceptable carrier is a dietary supplement or food. Examples of food that can be used to deliver a composition comprising recombinant bacterial spores include, but are not limited to, baby formula, yogurt, milk cheese, kefir, sauerkraut, and chocolate.

The present disclosure is also directed to methods of inducing tolerance/reducing sensitivity to allergens using compositions of recombinant bacterial spores.

“Oral” or “peroral” administration refers to the introduction of a substance, such as a vaccine, into a subject's body through or by way of the mouth and involves swallowing or transport through the oral mucosa (e.g., sublingual or buccal absorption) or both.

“Oronasal” administration refers to the introduction of a substance, such as a vaccine, into a subject's body through or by way of the nose and the mouth, as would occur, for example, by placing one or more droplets in the nose. Oronasal administration involves transport processes associated with oral and intranasal administration.

“Parenteral administration” refers to the introduction of a substance, such as a vaccine, into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

Liquid dosage forms for oral administration include pharmaceutically acceptable emulsions, microemulsions, solutions, suspensions, syrups and elixirs. In addition to a composition comprising recombinant bacterial spores, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to a composition comprising recombinant bacterial spores, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions comprising recombinant bacterial spores can be alternatively administered by aerosol. For example, this can be accomplished by preparing an aqueous aerosol, liposomal preparation or solid particles containing a composition comprising recombinant bacterial spores preparation. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants, innocuous proteins like serum albumin, sorbitan esters, oleic acid, lecithin, amino acids such as glycine, buffers, salts, sugars or sugar alcohols. Aerosols generally are prepared from isotonic solutions.

Compositions comprising recombinant bacterial spores can be alternatively administered by ingestion of food containing a composition comprising recombinant bacterial spores.

The amount of recombinant bacterial spores to be effective will depend upon, for example, the activity, the particular nature, pharmacokinetics, pharmacodynamics, and bioavailability of a particular vaccine preparation, physiological condition of the subject (including race, age, sex, weight, diet, disease type and stage, general physical condition, responsiveness to a given dosage and type of medication), the nature of pharmaceutically acceptable carriers in a formulation, the route and frequency of administration being used, to name a few. However, the above guidelines can be used as the basis for fine-tuning the treatment, e.g., determining the optimum dose of administration, which will require no more than routine experimentation consisting of monitoring the subject and adjusting the dosage. Remington: The Science and Practice of Pharmacy (Gennaro ed. 20th edition, Williams & Wilkins Pa., USA (2000)).

In one embodiment, the vaccine composition comprises about 1×10¹, 1×10², 1×10³, 1×10⁴, 1×10⁵, 1×10⁶, 1×10⁷, 1×10⁸, 1×10⁹ or more recombinant bacterial spores per administration dose.

The engineered (recombinant) bacterial cells disclosed herein are induced to form spores using methods known in the art, and the spores are administered to the animal to be treated. CTB and peanut antigens are expressed by the ingested bacterial spores and come into contact with the animal's immune system via the intestinal mucosa.

In one embodiment, the antigens are expressed on the surface of the orally administered spores, so that the antigens come into contact with the immune system (generally, lymphocytes in the blood or mucosa) of the animal upon ingestion. The antigens are expressed on the spore surface, individually or as a fusion protein, preferably together with a spore coat protein. If an antigen is expressed on the surface of spores, it can exert its immunogenic effects without germination of the spores. For example, an immune response can be elicited from the animal if the antigens contact or are taken up by cells in the mucosa, such as M cells.

In an alternate embodiment, the spores germinate in the host animal after ingestion, and replicate as vegetative bacterial cells which express and produce the recombinantly encoded antigen(s).

In either alternative, the antigens come into contact with the cells of the host animal and elicit an immune response.

In some embodiments, a composition disclosed herein is administered to a subject once a week, twice a week, three times a week or once every fortnight, once every three weeks or once a month. In some embodiments, the composition is administered multiple times, e.g., once, twice, three times, four times, five times, six times, seven times or eight times. In a specific embodiment, the composition is administered 3-8 times. In another embodiment, a booster dose of the composition is administered at least a month, at least two months, at least three months or at least six months from the initial or the last administered dose.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

Materials and Methods

Construction of Gene Fusions

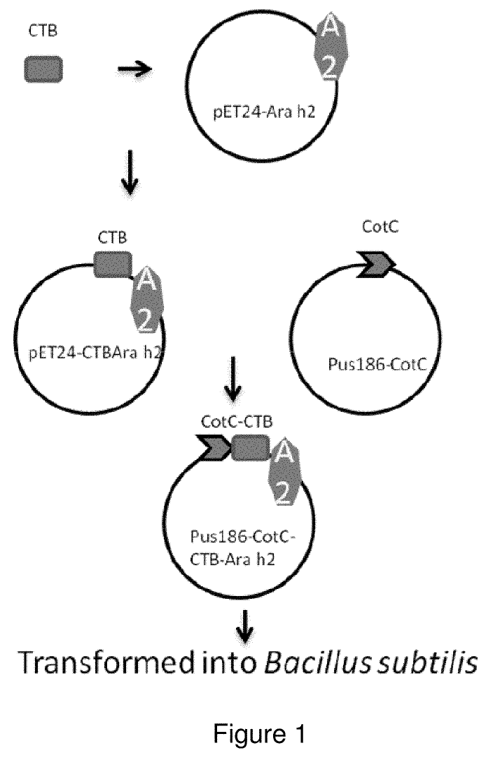

CTB DNA was amplified by PCR using the synthesized CTB DNA (GenScript, Piscataway, N.J.) as template and the following designed primers. The designed primers include: forward primer: 5′CGGGCTAGCACACCTCAAAATATTACTGAT3′ with a NheI site (underlined) (SEQ ID NO: 1), reverse primer: 5′GGCGAATTCATTTGCCATACTAATTGCG3′ with an EcoRI site (SEQ ID NO: 2). The PCR conditions were as follows: 94° C. for 4 m followed by 35 cycles of 94° C. for 30 s, 55° C. for 30 s and 72° C. 60 s, and the reaction continued for 10 min at 72° C. after the last cycle. The purified PCR product was digested with NheI, EcoRI and cloned into NheI/EcoRI double digested pET 24-Arah2 plasmid (provided by Dr. Hugh Sampson) and transformed to E. coli BL21. CTB-Arah2 DNA was amplified by using the constructed pET24-CTB-Arah2 plasmid as template. The PCR primers include: forward primer: 5′CGGTCTAGAGACACCTCAAAATATTACTGATT3′ with an XbalI site (SEQ ID NO: 3), reverse primer: 5′AAAAAGCTTTTAGTCTCTGTCTCTGCCGCCAC3′ with a HindIII site (SEQ ID NO: 4). The purified PCR product was double digested by XbalI/HindIII and ligated to the 3′ end of the CotC gene in pUS186-CotC plasmid construct (Zhou et al., (2008), Vaccine, 26, 1817-1825; Zhou et al., (2008), Parasitol Res, 102, 293-297) and transformed into B. subtilis WB600. See FIG. 1. The integrities of the fusion genes were confirmed by sequencing.

Gene Cloning Strategies for CTB-Ara h8:

The Arah8 coding gene was amplified by PCR using synthesized Arah8 sequence (SEQ ID NO: 8) as template (Huada gene). The designed primers included a forward primer (5′-AAAGTCGACATGGGCGTCTTCACTTTCGA-3′) (SEQ ID NO: 9) and a reverse primer (5′-GGCGCGGCCGCCTAATATTGAGTAGGGTTG-3′) (SEQ ID NO: 10), with restriction sites for SalI and NotI allowing amplified DNA to be cloned into the pET28a expression plasmid (Merck, Darmstadt, Germany). The CTB coding region (SEQ ID NO: 7) was cloned into the recombinant plasmid pET28a Arah8 to produce recombinant plasmid pET28a CTB-Arah8 with forward (5′-CGAGAATTCACACCTCAAAATATTACTGAT-3′) (SEQ ID NO: 11) and reverse (5′-CGAGTCGACATTGCCATACTAATTG-3′) (SEQ ID NO: 12) primers with restriction sites EcoRI, and SalI, respectively. All recombinant plasmids were identified by restriction endonuclease digestion analysis and DNA sequencing.

CTB-Arah8 DNA was amplified using the constructed pET28-CTB-Arah8 plasmid as template. The PCR primers included a forward primer (5′-CGCTCTAGACACACCTCAAAATATTACTG-3′) (SEQ ID NO: 13) with an XbalI restriction site and a reverse primer (5′-AAACTGCAGCTAATATTGATGAGGGTTGGC-3′) (SEQ ID NO: 14) with a PstI restriction site. The purified CTB-Arah8 PCR product was double digested by XbalI and PstI restriction enzymes, and cloned into the 3′ terminal of the CotC in the recombinant pUS186-CotC plasmid. This recombinant plasmid was then transformed into B. subtilis WB600 cells and confirmed by XbalI/PstI double enzyme digestion and DNA sequencing. Recombinant Pus186cotC-CTB-Ara h8 plasmid sequence is shown as following:

(SEQ ID NO: 15)
TATATACGGTCAAAAAAACGTATTATAAGAAGTATTACGAATATGATAAA
AAAGATTATGACTGTGATTACGACAAAAAATATGATGACTATGATAAAAA
ATATTATGATCACGATAAAAAAGACTATGATTATGTTGTAGAGTATAAAA
AGCATAAAAAACACTACCGTCTAGACACACCTCAAAATATTACTGATTTG
TGTGCAGAATACCACAACACACAAATACATACGCTAAATGATAAGATATT
TTCGTATACAGAATCTCTAGCTGGAAAAAGAGAGATGGCTATCATTACTT
TTAAGAATGGTGCAACTTTTCAAGTAGAAGTACCAGGTAGTCAACATATA
GATTCACAAAAAAAAGCGATTGAAAGGATGAAGGATACCCTGAGGATTGC
ATATCTTACTGAAGCTAAAGTCGAAAAGTTATGTGTATGGAATAATAAAA
CGCCTCATGCGATTGCCGCAATTAGTATGGCAAATGTCGACATGGGCGTC
TTCACTTTCGAGGATGAAATCACCTCCACCGTGCCTCCGGCCAAGCTTTA
CAATGCTATGAAGGATGCCGACTCCATCACCCCTAAGATTATTGATGACG
TCAAGAGTGTTGAAATTGTTGAGGGAAACGGTGGTCCCGGAACCATCAAG
AAACTCACCATTGTCGAGGATGGAGAAACCAAGTTTATCTTGCACAAGGT
GGAGTCAATAGATGAGGCCAACTATGCATACAACTACAGCGTTGTTGGAG
GAGTGGCTCTGCCTCCCACGGCGGAGAAGATAACATTTGAGACAAAGCTG
GTTGAAGGACCCAACGGAGGATCCATTGGGAAGCTTACTCTCAAGTACCA
CACCAAAGGAGATGCAAAGCCAGATGAGGAAGAGTTGAAGAAGGGTAAGG
CCAAGGGTGAAGGTCTCTTCAGGGCTATTGAGGGTTACGTTTTGGCCAAC
CCTACTCAATATTAGCTGCAGGCATGCAAGCTTTAATGCGGTAGTTTATC
ACAGTTAAATTGCTAACGCAGTCAGGCACCGTGTATGAAATCTAACAATG
CGCTCATCGTCATCCTCGGCACCGTCACCCTGGATGCTGTAGGCATAGGC
TT.

CTB-Arah8 DNA was amplified using the constructed pET28-CTB-Arah6 plasmid as template. The PCR primers included a forward primer (5′-CGGTCTAGACACACCTCAA AATATTACTG-3′) with an XbalI restriction site (SEQ ID NO: 86) and a reverse primer (5′-AATCTGCAGT TAGCATCTGCCGCCACT-3′) with a PstI restriction site (SEQ ID NO: 87). The purified CTB-Arah6 PCR product was double digested by XbalI and PstI restriction enzymes, and cloned into the 3′ terminal of the CotC in the recombinant pUS186-CotC plasmid. This recombinant plasmid was then transformed into B. subtilis WB600 cells and confirmed by XbalI/PstI double enzyme digestion and DNA sequencing.

Gene Cloning Strategies for Ara h6:

The Arah6 coding gene was amplified by PCR using synthesized Arah 6 sequence (SEQ ID NO: 16) as template (Huada gene). The designed primers included a forward primer (5′-AAAGTCGACATGGCCAAGTCCACCATCC-3′) (SEQ ID NO: 17) and a reverse primer (5′-AAAGCGGCCGCTTAGCATCTGCCGCCACT3′) (SEQ ID NO: 18), with restriction sites for SalI and NotI allowing amplified DNA to be cloned into the pET28a expression plasmid (Merck, Darmstadt, Germany). The CTB coding region was cloned into the recombinant plasmid pET28a Arah6 to produce recombinant plasmid pET28a CTB-Arah6 with forward (5′-CGGGAATTCACACCTCAAAATATTACTGAT-3′) (SEQ ID NO: 19) and reverse (5′-AAGGTCGACATTTGCCATACTAATTGCG-3′) (SEQ ID NO: 20) primers with restriction sites EcoRI, and SalI, respectively. All recombinant plasmids were identified by restriction endonuclease digestion analysis and DNA sequencing.

Gene Cloning Strategies for CTB-Ara h1 &3:

The epitope Ara h1&3 coding gene was amplified by PCR using synthesized epitope Ara h1(SEQ ID NO: 21) & Ara h3 (SEQ ID NO: 27) sequences as template (Huada gene). The designed primers included a forward primer (5′-AAAGTCGACGCCAAGTCATCACCT-3′) (SEQ ID NO: 22) and a reverse primer (5′-AAAGCGGCCGCTTAGCCACGCCT-3′) (SEQ ID NO: 23), with restriction sites for SalI and NotI allowing amplified DNA to be cloned into the pET28a expression plasmid (Merck, Darmstadt, Germany).

The CTB coding region was cloned into the recombinant plasmid pET28a epitope Ara h1&3 to produce recombinant plasmid pET28a CTB-epitope Ara h1&3 epitope Ara h1&3 with forward (5′-CGGGAATTCACACCTCAAAATATTACTGAT-3′) (SEQ ID NO: 19) and reverse (5′-AAGGTCGACATTTGCCATACTAATTGCG-3′) (SEQ ID NO: 20) primers with restriction sites EcoRI, and SalI (underlined) respectively. All recombinant plasmids were identified by DNA sequencing.

CTB-epitope Ara h1&3 DNA was amplified using the constructed pET28-CTB-epitope Ara h1&3 plasmid as template. The PCR primers included a forward primer (5′-CGGTCTAGACACACCTCAAAATATT-3′) (SEQ ID NO: 24) with an XbalI restriction site and a reverse primer (5′-AAACTGCAGTTAGCCACGCCT-3′) (SEQ ID NO: 25) with a PstI restriction site. The purified CTB-epitope Ara h1&3 PCR product was double digested by XbalI and PstI restriction enzymes, and cloned into the 3′ terminal of the CotC in the recombinant pUS186-CotC plasmid. This recombinant plasmid was then transformed into B. subtilis WB600 cells and confirmed by DNA sequencing. The recombinant pus186cotC-CTB-epitope Ara h1&3 plasmid sequence is shown as following:

(SEQ ID NO: 26)
CTTTCTATGATTTTAACTGTCCAAGCCGCAAAATCTACTCGCCGTATAAT
AAAGCGTAGTAAAAATAAAGGAGGAGTATATATGGGTTATTACAAAAAAT
ACAAAGAAGAGTATTATACGGTCAAAAAAACGTATTATAAGAAGTATTAC
GAATATGATAAAAAAGATTATGACTGTGATTACGACAAAAAATATGATGA
CTATGATAAAAAATATTATGATCACGATAAAAAAGACTATGATTATGTTG
TAGAGTATAAAAAGCATAAAAAACACTACCGTCTAGACACACCTCAAAAT
ATTACTGATTTGTGTGCAGAATACCACAACACACAAATACATACGCTAAA
TGATAAGATATTTTCGTATACAGAATCTCTAGCTGGAAAAAGAGAGATGG
CTATCATTACTTTTAAGAATGGTGCAACTTTTCAAGTAGAAGTACCAGGT
AGTCAACATATAGATTCACAAAAAAAAGCGATTGAAAGGATGAAGGATAC
CCTGAGGATTGCATATCTTACTGAAGCTAAAGTCGAAAAGTTATGTGTAT
GGAATAATAAAACGCCTCATGCGATTGCCGCAATTAGTATGGCAAATGTC
GACGCCAAGTCATCACCTTACCAGAAGAAAACACTCGAGTATGATCCTCG
TTGTGTCTATGATGGGGAGCGGACACGTGGCCGCCAACCCGGACGTAGGT
ACACAGCGAGGTTGAAGGAAGGCGGAAACATCTTCAGCGGCTTCACGCCG
GAGTTCCTGGAACAAGCCGTGACAGTGAGGGGAGGCCTCAGAATCTTGAG
CCCAGATAGAAAGGATGAAGATGAATATGAATACGATGAAGAGGATAGAA
GGCGTGGCTAACTGCAGGCATGCAAGCTTTAATGCGGTAGTTTATCACAG
TTAAATTGCTAACGCAGTCAGGCACCGTGTATGAAATCTAACAATGCGCT
CATCGTCATCCTCGGCACCGTCACCCTGGATGCTGTAGGCATAGGCTTGG
TTATGCCGGTACTGCCGGGCCTATTTCACTTTTTGCATTCTACAAACTGC
ATAACTCATATGTAAATCGCTCCTTTTTAGGTGGCACAAATGTGAGGCAT
TTTCGCTCTTTCCGGCAACCACTTCCAAGTAAAGTATAACACACTATACT
TTATATTCATAAAGTGTGTGCTCTGCGAGGCTGTCGGCAGTGCCGACCAA
AACCATAAAACCTTTAAGACCTTT

Epitope CTB-A1 &3 defined above was made up of Ara h1 epitope peptides 1, 3, 4, and 17 (SEQ ID NOs: 28, 30, 32 and 34) listed in Table 1 and Ara h3 epitope peptides 2, 3 and 4 (SEQ ID NOs: 36, 38 and 40) listed in Table 3.

Epitopes from Ara h1

The following peptides from the peanut antigen Ara h1 were used in the exemplary embodiments of the present invention:

Peptide with the sequence AKSSPYQKKT (SEQ ID NO: 28) which can be encoded by the nucleotide sequence: GCCAAGTCATCACCTTACCAGAAGAAAACA (SEQ ID NO: 29);

Peptide with the sequence LEYDPRLUYD (SEQ ID NO: 30) which can be encoded by the nucleotide sequence: CTCGAGTATGATCCTCGTTGTGTCTATGAT (SEQ ID NO: 31);

Peptide with the sequence GERTRGRQPG (SEQ ID NO: 32) which can be encoded by the nucleotide sequence: GGGGAGCGGACACGTGGCCGCCAACCCGGA (SEQ ID NO: 33);

Peptide with the sequence RRYTARLKEG (SEQ ID NO: 34) which can be encoded by the nucleotide sequence: CGTAGGTACACAGCGAGGTTGAAGGAAGGC (SEQ ID NO: 35).

Epitopes from Ara h3

Following peptides from the peanut antigen Ara h3 were used in the exemplary embodiments of the present invention (see Table 3):

Peptide with the sequence GNIFSGFTPEFLEQA (SEQ ID NO: 36) which can be encoded by the nucleotide sequence:

(SEQ ID NO: 37)
GGAAACATCTTCAGCGGCTTCACGCCGGAGTTCCTGGAACA
AGCC;

Peptide with the sequence VTVRGGLRILSPDRK (SEQ ID NO: 38) which can be encoded by the nucleotide sequence:

(SEQ ID NO: 39)
GTGACAGTGAGGGGAGGCCTCAGAATCTTGAGCCCAGATAGA
AAG;

Peptide with the sequence DEDEYEYDEEDRG (SEQ ID NO: 40) which can be encoded by the nucleotide sequence:

(SEQ ID NO: 41)
GATGAAGATGAATATGAATACGATGAAGAGGATAGAAGGCG
TGGC.

Spore Coat Protein Extraction and Western Blot Analysis

Pus186cotC-CTB-Ara h2/B. subtilis WB600 strain was cultured in LB medium with 25 μg/ml kanamycin at 37° C. overnight, and then transferred to Difco Sporulation Medium (DSM) and cultured for 24 hours for sporulation. Spores were collected and purified as previously described (Zhou et al., (2008), Vaccine, 26, 1817-1825). Briefly, the spores were incubated with 4 mg/ml lysozyme followed by washing in 1 M NaCl and 1 M KCl with 1 mM PMSF. After the last suspension in water, spores were treated at 65° C. for 1 h in water bath to kill any residual sporangial cells. Spore numbers were determined by direct counting under microscopy by using hemacytometer. Approximately 10¹¹ spores were obtained from 1.0 L of DSM medium.

Spore coat proteins were extracted from suspensions of spores at high density (>1×10¹⁰ spores per ml) in sodium dodecyl sulphate-dithiothreitol (SDS-DTT) extraction buffer (0.5% SDS, 0.1 M DTT, 0.1 M NaCl) by sonication. To confirm the surface display of CTB-Ara h2 on the spores coat, extracted proteins were separated on a 12% SDS-PAGE gel and then transferred onto a nitrocellulose membrane. Proteins were incubated with mouse anti-Ara h2 antibody, reactive bands were visualized with horseradish peroxidase (HRP)-coupled anti-mice antibody via Chemiluminescent HRP Antibody Detection Reagent (Denville Scientific, South Plainfield, N.J.) according to the manufacturer's procedures.

Mice Model and Treatment

Five-week-old female C3H/HeJ mice purchased from Jackson Laboratory (Bar Harbor, Me.) were maintained on peanut-free chow under specific pathogen-free conditions according to standard guidelines for the care and use of animals (Institute of Laboratory Animal Resources Commission of Life Sciences NRC. 1996). There were 15 mice in three groups: sham, rCTB-Ara h2 spores treatment and naïve.

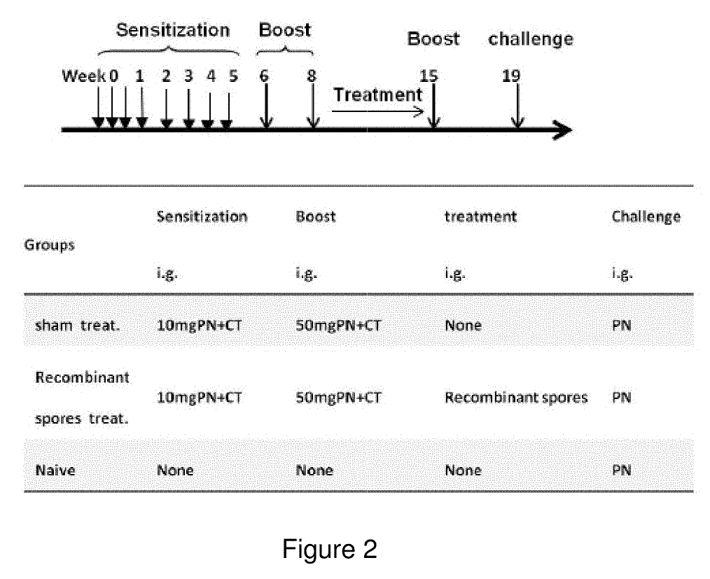

Roasted peanuts were shelled with red skins retained, and allowed to soak in PBS for 20 minutes, peanuts were blended periodically in phosphate-buffered saline (PBS) for 3 h until a smooth suspension was obtained. Mice were sensitized intragastrically with peanut (10 mg) and cholera toxin (20 μg; List Laboratories Campbell, Calif.) in a total volume of 500 μL PBS on 3 consecutive days of week 0, and once a week from weeks 1-5. Mice were boosted at weeks 6, 8 and 15 with 50 mg peanut and 20 μg cholera toxin. Mice were administrated orally by intragastric lavage with 1.0×10⁹ rCTB-Arah2 spores in 0.5 ml volume for 3 consecutive days weekly from week 9 to week 14 and challenged 4 weeks post therapy (FIG. 2).

Assessment of Hypersensitivity Reactions

Anaphylactic symptoms were evaluated 30 minutes after oral challenge using the following scoring system: 0 no reaction; 1 scratching and rubbing around the snout and head (mild); 2 puffiness around the eyes and snout, diarrhea, pilar erection, reduced activity, and/or decreased activity with increased respiratory rate (moderate); 3 wheezing, labored respiration, cyanosis around the mouth and the tail (severe); 4 no activity after prodding, or tremor and convulsion (near fatal); and 5 death. Core body temperatures were measured using a rectal probe (Harvard Apparatus, Holliston, Mass.).

Measurement of Peanut Specific Immunoglobulin

Blood was collected by submandibular venipuncture and harvested sera were stored at −80° C. until needed. For Peanut-specific IgE, 100 μl 500 μg/ml CPE was used to coat wells overnight at 4° C., 1:20 dilution of sample was added to coated well and incubated at 4° C. overnight. In the third day, 1 μg/ml biotinylated rat anti-mouse IgE antibody (BD, San Diego, Calif.) was added to each well and incubated for 1 h at room temperature, followed by adding avidin-HRP (Sigma, Louis, Mo.) and incubated for 45 m at room temperature. Signals were detected by TMB substrate reagent (BD, San Diego, Calif.). The peanut specific serum IgA was measured by the similar protocol above except for using 20 μg/ml CPE to coat the wells and biotinylated rat anti-mouse IgA antibody as detection antibody. For peanut specific IgG1 and IgG2a measurement, 20 μg/ml crude peanut extract was used to coat plates and the sample dilutions were 1:4000, 1:40000 respectively.

Histamine Measurement

Blood was collected 30 minutes after peanut challenge using EDTA tube (BD, Franklin Lakes, N.J.) and chilled on ice immediately. Plasma was isolated by centrifuging at 900 g for 10 m at 4° C. within 20 m of sample collection. Histamine levels were measured using an enzyme immunoassay kit (Immunotech Inc., Marseille, France) as described by the manufacturer. Briefly, 100 μl 1:150 dilution samples mixed with acylation reagent, 50 μl acylated samples (including calibrator and control) was added to antibody coated wells with 200 μl conjugate incubated 2 h at 4° C. while shaking. Substrate was added and the absorbance was read at 405 nm.

Cell Culture and Cytokine Measurements

Splenocytes were isolated from spleens removed from each group of mice, which had been sacrificed immediately after evaluation of the anaphylactic reactions, and cultured in RPMI 1640 containing 10% FBS, 1% penicillin/streptomycin, and 1% glutamine. Splenocytes were cultured in 24-well plates (4×10⁶/well/ml) in the presence or absence of CPE (200 m/ml). Supernatants were collected after 72 h of culture and aliquots were stored at −80° C. until analyzed. IL-4 and IL-10 levels were determined by ELISA according to the manufacturer's instructions (BD PharMingen)

Flow Cytometry Measurements of T_{reg} Cells

Splenocytes (SPCs) were obtained after 72 hours of culture and identification and quantification of T_{reg}s was determined by flow cytometry as previously described. Briefly, 4×106 cells were incubated in 1000 of staining buffer (2% BSA in 1×PBS) and 20 μg/ml of purified anti-CD16/32 mAb (2.4G2) as Fcγ receptor-blocking mAb for 30 minutes at 4OC. FITC-conjugated anti-mouse CD4, APC-conjugated anti-mouse CD25 were then added to the cell suspension in the presence of Fcγ receptor blocking mAb on ice for 30 minutes in the dark. After washing, cells were acquired on an LSR-II flow cytometer (BD Bioscience, Calif.) and data was analyzed using Flowjo software (Tree Star, Inc. Ashland, Oreg.)

Statistical Analysis

All statistical analyses were performed using Graphpad Prism4 software (GraphPad Software, La Jolla, Calif.). Differences between multiple groups were analyzed by one-way ANOVA followed by Dunnett's Multiple Comparison Test. A p-value ≦0.05 was considered to be statistically significant.

### Example 1: Expression of CTB-Ara h2 in the Spores Coat

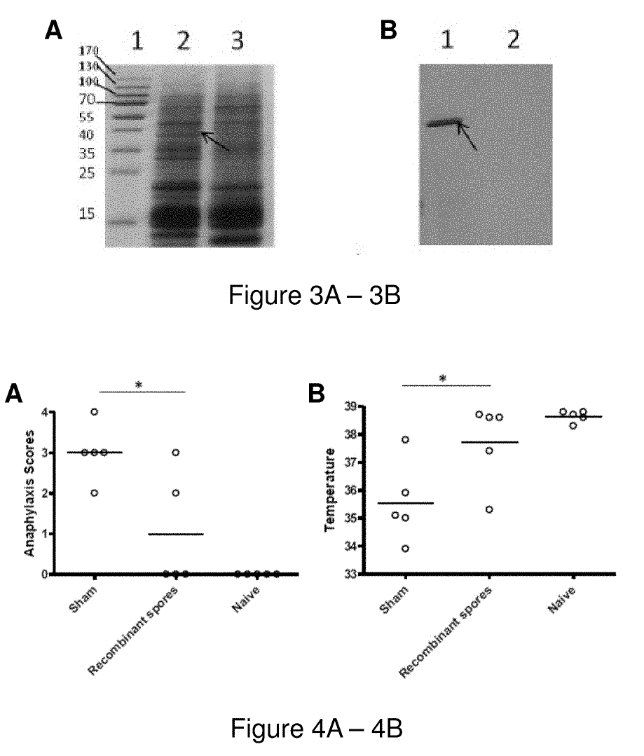

Recombinant plasmid of pus186-CotC-CTB-Ara h2 was transformed into B. subtilis WB600 and sporulation was formed in DSM using exhaustion method. SDS-PAGE showed that there was an objective band in the recombinant spores coat extraction as the molecular weights was about 37.1 kD corresponding to the CotC (8.8 kD) plus CTB (11.6 kD) and Ara h2 (16.7 kD) which the non-recombinant spores was absent (FIG. 3B). Western blotting with Ara h2 antibody also showed positive band of approximate 37.1 kD in spores of recombinant strains (FIG. 3B).

### Example 2: Oral Administration of rCTB-Ara h2 Spores Modulate Peanut Specific Immunoglobulin

Prior to treatment, the peanut-specific IgE levels in peanut-allergic mice were all elevated after 8 weeks sensitization. The peanut-specific IgE levels in rCTB-Ara h2 spores treated mice (1.0×10⁹) week 12) were significantly decreased compared with the IgE level before treatment (week 8) (p<0.05). In contract, peanut-specific IgE levels in sham mice at week 12 were not significantly different from that of week 8 (P>0.05) (Table 7). It showed that 4 weeks rCTB-Ara h2 spores treatment could significantly reduce the mice peanut IgE.

Peanut specific IgA level in the treated group mice at week 12 was significantly increased compared with sham mice (P<0.01) (Table 7). Compared with the mice before treatment (week 8), the treated group peanut specific IgA levels (12 W, 14 W) was also significantly increased (P<0.01). In contrast, there were no significant differences between that of sham mice (P>0.05).

TABLE 7
Serum peanut specific immunoglobulin levels

| | | Prior to | | | |
|---|---|---|---|---|---|
| | | Treatment | During Treatment | | |
| Immunoglobulin | Groups | 8 W | 10 W | 12 W | 14 W |
| IgE(ng/ml) | Sham | 1524 ± 900 | 1505 ± 1236 | 1005 ± 687 | 1042 ± 748 |
| | recombiant spores | 1654 ± 730 | 1071 ± 968 | 554 ± 273* | 512 ± 274* |
| | Naïve | 50 ± 5 | 59 ± 12 | 45 ± 10 | 55 ± 11 |
| | p, spore vs Sham | >0.05 | >0.05 | <0.05 | <0.05 |
| IgA(ng/ml) | Sham | 229 ± 146 | 245 ± 46 | 262 ± 36 | 174 ± 27 |
| | recombiant spores | 213 ± 101 | 317 ± 48 | 464 ± 75** | 453 ± 96** |
| | Naïve | 20 ± 3 | 18 ± 4 | 16 ± 3 | 19 ± 3 |
| | p, spore vs Sham | >0.05 | >0.05 | <0.01 | <0.01 |
| IgG1(ug/ml) | Sham | 1012 ± 498 | 273 ± 213 | 142 ± 178 | 128 ± 145 |
| | recombiant spores | 698 ± 91 | 523 ± 342 | 337 ± 233 | 344 ± 212 |
| | Naïve | 0 | 0 | 0 | 0 |
| | p, spore vs Sham | >0.05 | >0.05 | >0.05 | >0.05 |
| IgG2a(ug/ml) | Sham | 649 ± 323 | 227 ± 349 | 230.2 ± 408.8 | 172 ± 274 |
| | recombiant spores | 375 ± 197 | 356 ± 276 | 318 ± 227 | 488 ± 418 |
| | Naïve | 0 | 0 | 0 | 0 |
| | p, spore vs Sham | >0.05 | >0.05 | >0.05 | >0.05 |

There was an increased trend in peanut specific IgG2a in rCTB-Ara h2 spores treated mice in 14 week, but no significant difference compared with sham mice (P>0.05). The peanut specific IgG1 in sham and rCTB-Ara h2 spores treated mice decreased gradually with time, but there was no significant difference between sham and recombinant treated mice (P>0.05) (Table 7).

Oral administration of rCTB-Ara h2 spores reduces hypersensitivity reactions following peanut challenge. The mice in sham group all developed symptoms after peanut challenge in week 19, 1 mice score 4, 3 mice score 3, 1 mice score 2. In contrast, in rCTB-Ara h2 spores treated mice, only two mice developed symptoms, 1 mice score 3, 1 mice score 2. The symptom scores were significantly reduced in rCTB-Ara h2 spores treated group compared with sham group (P<0.05) (FIG. 4A).

Decreased core body temperature correlates with the severity of systemic anaphylaxis. The mean temperature in sham group mice after peanut challenge significantly decreased compared with rCTB-Ara h2 spores treated mice (p<0.05) (FIG. 4B).

### Example 3: Oral Administration of rCTB-Ara h2 Spores Reduce Plasma Histamine Release Following Peanut Challenge

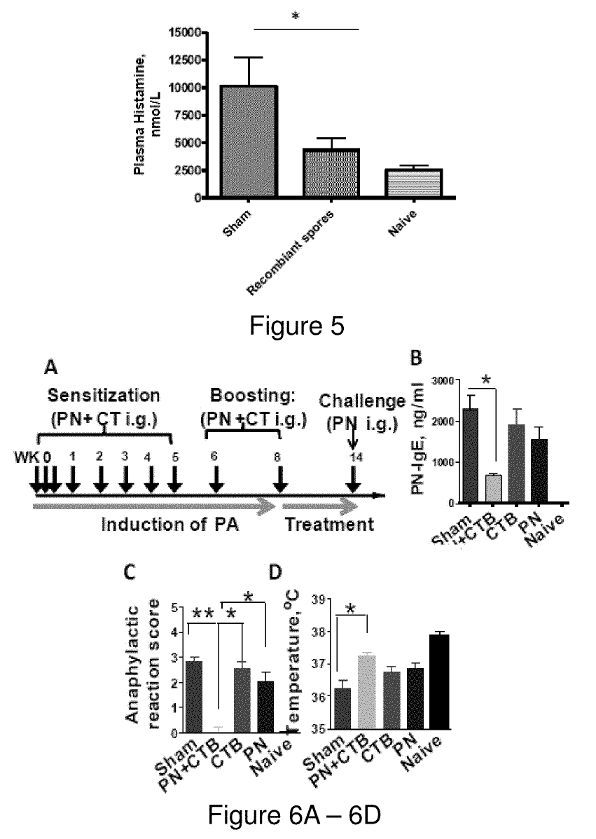

Plasma histamine levels of sham group mice were markedly increased 30 minutes after challenge compared with rCTB-Ara h2 spores treated mice (p<0.05) and naïve mice (p<0.01) (FIG. 5). The mean histamine level of rCTB-Ara h2 treated mice were not significantly different from naïve mice (P>0.05).

### Example 3: Peanut (PN) Mixed with CTB (PN+CTB) Consumption Protects Against PN Anaphylaxis

In the first investigation of CTB as toleragenic adjuvant for peanut (PN) vaccine, female C3H/HeJ mice with PA,1, 2 received CTB+PN daily for 6 weeks beginning at week 8 (FIG. 6A). This treatment (green bars) significantly reduced PN specific IgE levels (PN sIgE), anaphylactic reaction scores using an established scoring system ranging from 0 (no reaction) to 5 (death), 3-Sand hypothermia as compared to Sham treated-mice (red bars) (FIG. 6B-6C p<0.05-p<0.01). PN alone and CTB alone (blue, violet bars) did not significantly reduce these parameters when compared with sham treated mice.

### Example 4: PN+CTB Consumption During Gestation and Lactation Protected Against PN Anaphylaxis and Induced Tolerogenic Immune Response in Mothers and Offspring

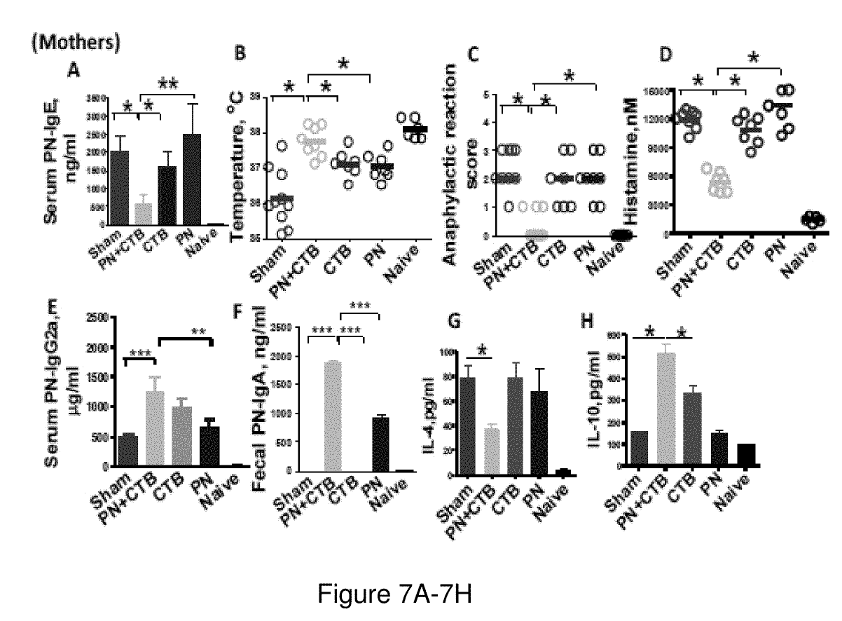

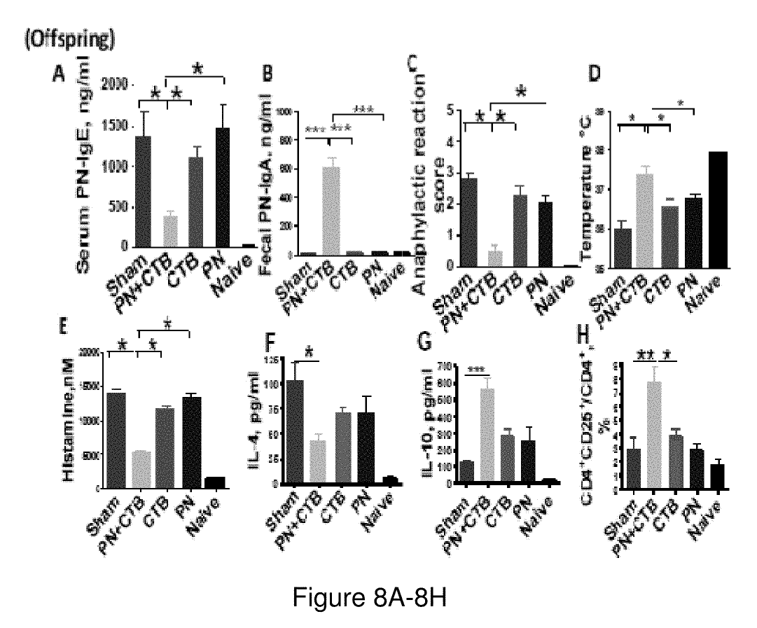

C3H/HeJ female mice were sensitized with PN as in FIG. 6A for 6 weeks. One week later, they were mated with naive males. During gestation and lactation (G/L), these Peanut Allergic Mice (PAMs) received oral PN+CTB, or PN, or CTB. Naïve mice were normal controls. After offspring weaning, mothers were challenged with PN. PN+CTB, but not PN or CTB alone during G/L significantly reduced serum PN specific sIgE, symptom scores and histamine release (FIGS. 7A-7D), and significantly increased IgG2a and fecal IgA levels (FIG. 7E-7F). Reduced IL-4, and increased IL-10 production by cultured SPCs (not shown) and mesenteric lymph node (MLN) cells was also found (FIG. 7G-7H). Five week old offspring of PAM fed PN+CTB, CTB alone or PN alone during G/L and naïve mothers were subjected to a PN sensitization regimen and challenged as in FIG. 6A. Offspring of Peanut Allergic Mother (OPAM) fed PN+CTB resistance to PN sensitization is shown by reduced serum PN-sIgE (FIG. 8A), increased serum sIgG2a (data not shown) and fecal PN sIgA (FIG. 8B), and greater protection at PN challenge (FIG. 8A-8D), reduced IL-4 and increased IL-10 production by SPCs and MLNCs (FIG. 8E-8G) as compared to OPAM receiving sham, PN alone or CTB alone treatment during G/L. These offspring's SPCs also contained significantly higher numbers of CD4+CD25+ T cells (FIG. 3H). These data demonstrate the importance of the mucosal adjuvant CTB in a PN vaccination regimen for inducing physiological and immunological tolerance in PAM and offspring.

### Example 4: Oral Vaccination with Recombinant BS Spores Surface Expressing CTB-Ara h2 Fusion Protein Vaccine (BCAV) Reduces PN Anaphylaxis, Increases Tolerogenic Immune Response, and Suppresses Th2

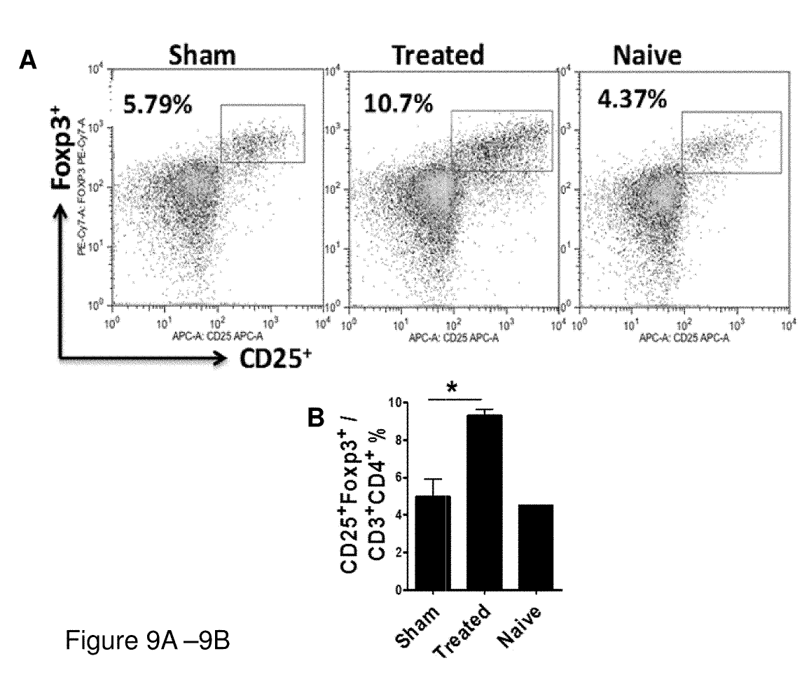

BS spores surface expressing CTB alone, and CTB plus constructs expressing Ara h2, Ara h1 or Ara h3 were generated. As found with PN+CTB, treatment with a mixture of BS spores surface expressing CTB and the 3 PN allergen constructs (named mixed spore constructs) significantly reduced PN sIgE and anaphylactic scores in the PA model at a 416 fold lower dose of PN protein than following PN+CTB treatment (6 μg vs 2500 μg). These mice were bred with naïve male mice, and 14 day fetal SPCs were collected and DNA methylation and IL-10 regulatory miRNA expression were determined. Beneficial epigenetic changes were found. Maternal splenocyte IL-10 production from mixed spore constructs treated mice was 64% higher than sham treated group SPCs. T_{reg} numbers were also increased (FIGS. 9A and 9B).

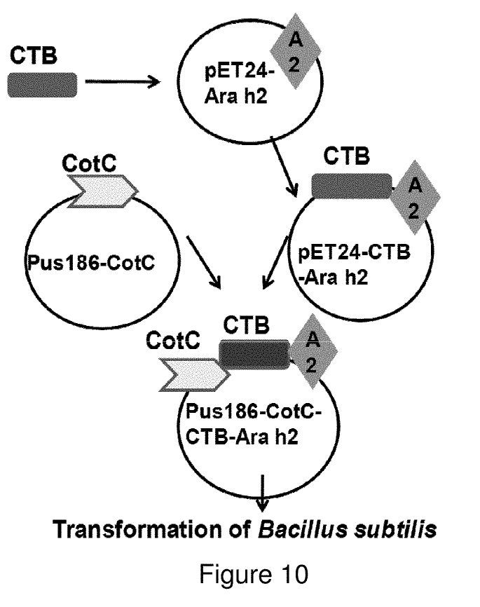

Next BS spores surface expressing CTB/Ara h2 fusion protein were generated, because CTB conjugated to antigen is markedly more potent than co-administration of Ag and CTB. A construct, (named BS-CTB-Ara h2 (BCAV), recombinant plasmid of Pus186-CotC-CTB-Ara h2 was generated using cloning, and transformed into BS WB600 (FIG. 10). Sporulation was induced in Difco sporulation medium (DSM) using the exhaustion method. SDS-PAGE and Western blotting with Ara h2 antibody showed the band in the recombinant spores coat extraction of molecular weights ˜37.1 kD corresponding to the CotC (8.8 kD) plus CTB (11.6 kD) and Ara h2 (16.7 kD), which was absent in the extract of non-recombinant spores. Next the established protocol described in FIG. 6A was used to determine BCAV prevention of PN anaphylaxis in dams. PA mice were fed. 1.0×10⁹ BCAV (equivalent to 2 μg recombinant CTB/Ara h2 fusion protein) in 0.5 ml PBS on 3 consecutive days weekly for 5 weeks (wks 9-wk 14) and challenged 4 weeks post therapy, BCAV treatment significantly reduced PA mice anaphylactic symptom scores, hypothermia, and histamine release, PN-specific IgE levels and increased PN specific IgA levels when compared with vehicle sham treated mice.

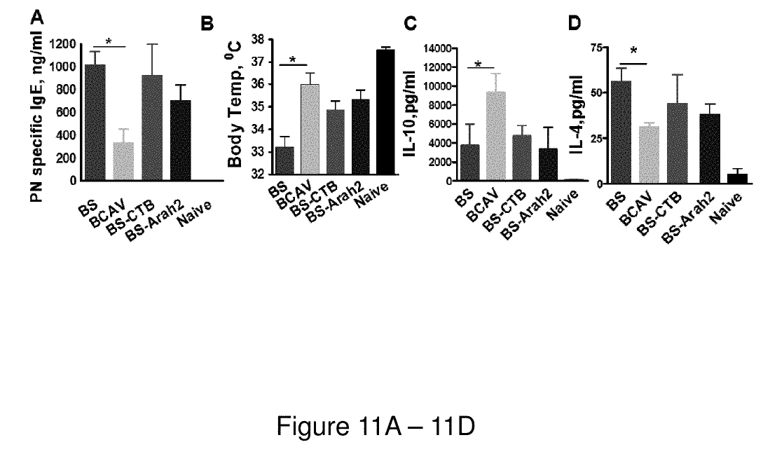

In a separate experiment, the effect of BCAV was compared to various control treatments in an epicutaneously (e.p.) PN sensitized model to mimic sensitization via skin contact in pediatric eczema patients. Mice were sensitized e.p. for 6 weeks followed by BCAV, or BS-Ara h2, BS-CTB (BS spores surface expressing Ara h2 alone or CTB alone) or BS spores alone for 4 weeks. BCAV was superior in suppressing PN sIgE production, preventing anaphylaxis, and suppressing SPC IL-4 production and increasing IL-10 production (FIGS. 11A-11D).

In summary, this proof of concept data shows that 1) Similar to PN+CTB treatment, BCAV suppresses PN anaphylaxis, reduces IgE and increases IgA levels and suppresses IL-4 and increases IL-10 production, at an approximately 1,250 fold lower dose of whole PN protein than PN+CTB (2 μg vs 2500 μg) and 116 fold lower dose of Ara h2 protein (2 vs 232 μg Ara h2.2). BCAV also produces sustained protection through at least 4 weeks post therapy, whereas PN protein alone treatment in the FA model resulted in only 2 weeks post therapy protection. 3). BCAV will be more cost effective than mixed BS constructs in potential future clinical studies (1 construct vs 4 constructs).

### Example 5: Use of a Novel Murine Model in which Maternal Ragweed-Induced Allergic Rhinitis (AR) Increases Offspring Peanut Allergy (PA) Risk

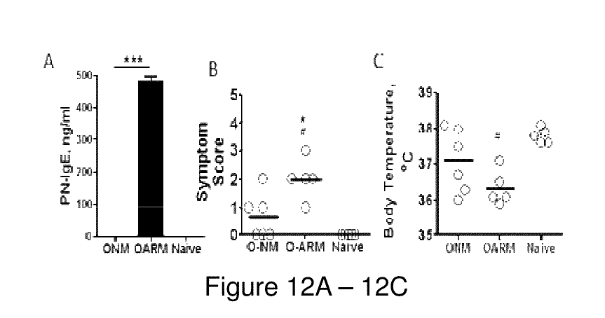

In 1996, Hourihane et al reported that the prevalence of PA increased in successive generations in maternal but not paternal relatives (Hourihane et al., BMJ, (1996); 313:518-21). Additional clinical observational studies also show that maternal peanut allergy and other allergies increase the risk for a child to develop peanut allergy (Lack G. et al., N Engl J Med (2003); 348:977-85). However, direct experimental evidence that maternal environmental allergies such as AR increases offspring PA risk, had not been demonstrated. Therefore, a ragweed-induced allergic rhinitis (AR) model was established. The AR mice exhibited sneezing and nasal rubbing symptoms, and eosinophils in nasal lavage fluids. As a result, Offspring of Allergic Rhinitis Mouse (O-ARM) showed significantly higher PN sIgE levels, anaphylactic symptoms and hypothermia following oral PN challenge (FIG. 12A-12C).

### Example 6: CTB+PN Alters DNA Methylation at IL-4 and Foxp3 Promoters in Maternal Peripheral Blood Leukocytes (PBL) and Offspring CD4+ T Cells

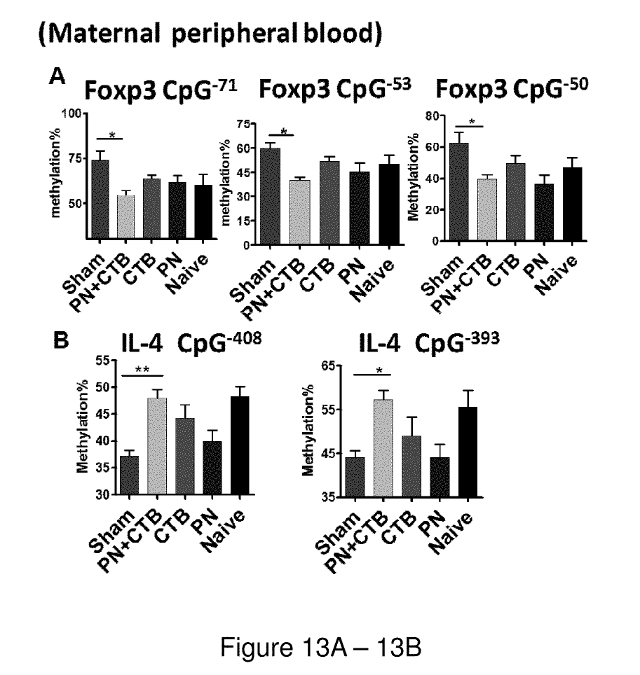

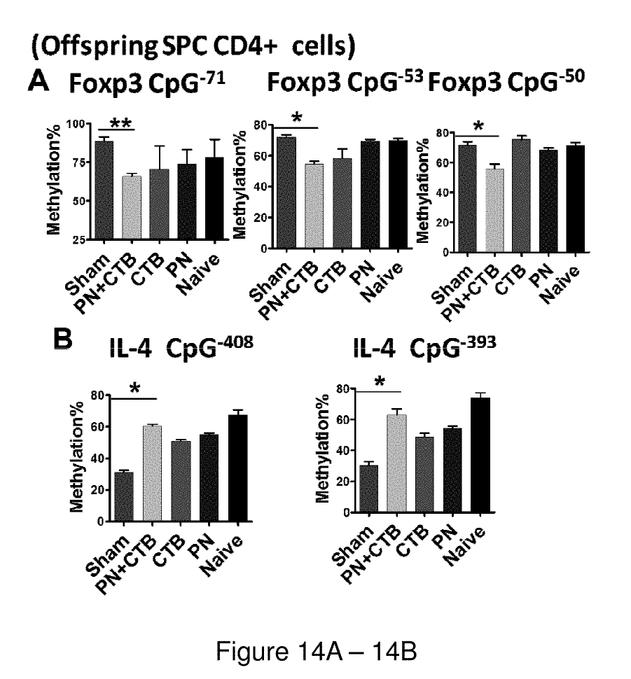

After finding that PN+CTB induced tolerance in PAM increased T_{reg} numbers in SPCs from young offspring and prevented PA, DNA methylation of Foxp 3 and IL-4 promoters in mother PBL and offspring CD4 T cells were determined. Genomic DNA extracted from PAM that received CTB+PN, PN alone, CTB alone, sham treatment or naïve mice from FIG. 7 were analyzed for DNA methylation levels of Foxp3 and IL-4 promoter. Methylation levels at CpG⁻⁷¹,CpG⁻⁵³ and CpG⁻⁵⁰ in the Foxp3 promoter were lower, indicating gene activation, and CpG residues (CpG⁻⁴⁰⁸, CpG⁻³⁹³) of the IL-4 promoter in SPC in PN+CTB mothers cells were higher indicating gene suppression than sham mothers (p<0.05-p<0.01, FIGS. 13A and 13B). These alterations did not occur in CTB alone and PN alone treated PAM cells. A similar pattern of DNA methylation of Foxp 3 and IL-4 promoters of offspring was found (FIGS. 14A and 14B).

### Example 7: BS Recombinant Spores Surface Expressing CTB and Individual PN Ags Alters miRNA Expression in Fetal SPCs

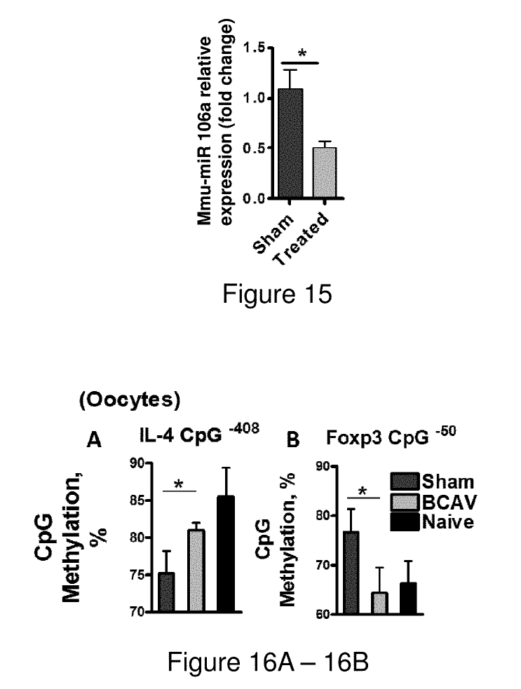

As the study progressed to generating BS spores surface expressing CTB and individual PN Ags, it was found that spore constructs increased IL-10 production 3 fold and doubled the number of T_{reg}s. As a first step to determine if the CTB based PN vaccine induces IL-10 via miRNA epigenetic regulation, and to establish methodology, expression of miR-106a, a negative regulator of IL-10 gene activation, was determined in fetal (16-18 day old) splenocytes from fetuses of PAM treated with mixed spore constructs. Interestingly, miR-106a expression was significantly lower in fetal splenocytes from mixed BS recombinant spores treated PAM than from sham treated PAM (FIG. 15).

### Example 8: Maternal Preconception BCAV Alters of DNA Methylation at IL-4 and Foxp3 Promoters in Oocytes

Next, DNA methylation status in oocytes were determined from the same vaccinated and control mice in FIG. 11. One week after PN maternal challenge, their oocytes were collected by Kevin Kelley, Ph.D, Director of the Mouse Genetics and Gene Targeting Core, Icahn School of School of Medicine at Mount Sinai. Increased methylation levels at IL-4 promoter CpG-408 in oocytes and increased methylation levels at IL-4 promoter CpG-408 and CpG-393 in peripheral blood were found.

In summary, these data demonstrate that PN+CTB and the more advanced PN vaccine—BCAV—induction of PN tolerance is associated with epigenetic modifications in DNA methylation on Foxp3 and IL-4 gene promoters and IL-10 regulator miRNA expression in mothers and offspring.

### Example. 9: Maternal BCA2 Vaccine Prevents Peanut Allergy Development and Induces Tolerogenic Immunity in High Risk Offspring

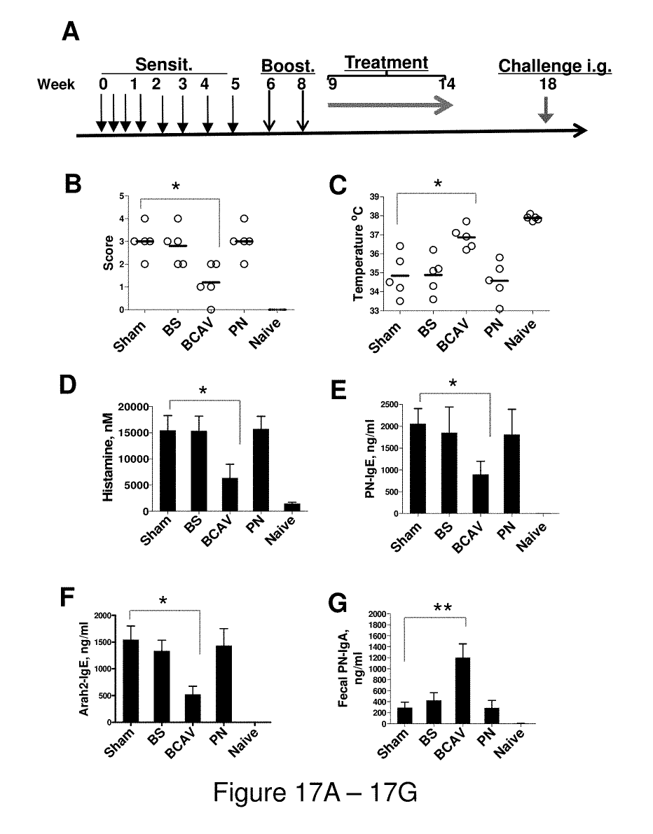

Oral administration of BCAV reduced hypersensitivity reactions following peanut challenge as shown in FIG. 17A. The mice in sham group all developed symptoms after peanut challenge in week 18, 1 mouse score 4, 3 mice score 3, 1 mouse score 2. In contrast, in BCAV treated mice, 2 mice were score 2, and 2 mice were score 1 and 1 mouse scored 0. The median symptom scores were reduced significantly in BCAV treated group compared with sham group (P<0.05) (FIG. 17B).

In addition, the mean temperature in sham group mice after peanut challenge decreased significantly compared with BCAV treated mice (p<0.05) (FIG. 17C). BS spores group and PN alone group didn't show significant differences with sham group regarding reaction scores and body temperature.

Oral administration of BCAV also reduced plasma histamine release following peanut challenge: Histamine release from mast cell and basophil degranulation is the major mechanisms underlying anaphylactic reactions. It was previously found that plasma histamine levels are correlated with the severity of anaphylactic reactions in this model. Therefore, effect of BCAV on plasma histamine levels was determined 30 min after challenge. Consistent with previous findings, plasma histamine levels of sham group mice were markedly increased 30 minutes after challenge compared with BCAV treated mice (p<0.05) and naïve mice (p<0.01)(FIG. 17D). The mean histamine level of BS and PN alone treated mice were not significantly different from sham mice (P>0.05).

Oral administration of BCAV modulated peanut specific immunoglobulin: The peanut-specific IgE levels in BCAV treated mice (week18) were significantly decreased significantly compared with the IgE level in sham mice, BS spore and PN alone treated mice. It showed that 5 weeks BCAV treatment could reduce the mice peanut IgE significantly (FIG. 17E). Similarly, the Arah2-specific IgE levels in BCAV treated mice (week18) were significantly decreased significantly compared with the IgE level in sham mice, BS spore and PN alone treated mice (FIG. 17F). Peanut specific IgA level in the BCAV treated group mice at week 18 was significantly increased compared with sham mice, BS spore and PN alone treated mice (FIG. 17G, P<0.05).

### Example 10. BCAV Induces PN-Specific IgE Reduction and IgA Increase in Offspring Born from these Mothers

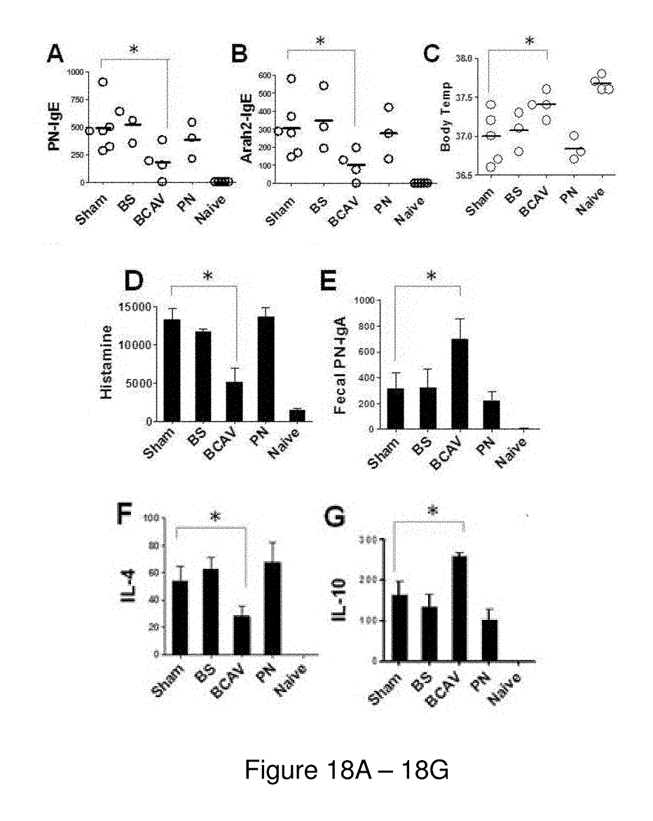

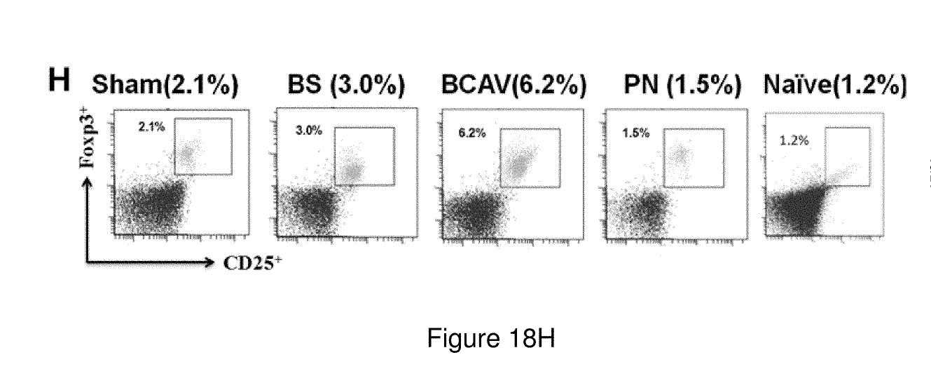

PN-specific IgE levels and Arah2-specific IgE levels were significantly lower in offspring of BCAV-fed PNA mothers (p<0.05, FIGS. 18A-18B). Offspring of PN alone or BS fed PNA mothers did not show significant difference in PN-specific IgE levels compared to offspring of sham fed PNA mothers. We also determined the immunoglobulins' levels in the offspring feces. Offspring of BCAV mothers had significantly higher PN-specific IgA levels than other groups (p<0.05; FIG. 18E).

BCAV vaccine protected offspring born from BCAV fed mothers against anaphylactic reaction following peanut oral challenge. Core body temperatures of sham-fed mothers' offspring were significantly lower than naïve mice, but normal in offspring of BCAV-fed mothers (FIG. 18C). Plasma histamine levels in offspring of BCAV-fed mothers were lower than those of sham-fed mothers' offspring (p<0.05, FIG. 18D). Offspring of PN alone or BS fed PNA mothers did not show significant difference in body temperature (FIG. 18C) and histamine levels (FIG. 18D) compared to offspring of sham fed PNA mothers. These results showed that feeding BCAV to PNA mothers during pregnancy and lactation provided significant protection to their offspring against PN anaphylaxis compared to offspring from sham-fed PNA mothers.

Reduction of Th₂ cytokines, increase T_{reg} cytokines and T_{reg} cells. To determine any association between the protective effects of BCAV on cytokine profiles in offspring, IL-4 and IL-10 production by splenocytes from each group of mice were measured. Significant decreases in IL-4 and increases IL-10 were found in BCAV fed mother's offspring (FIGS. 18F-18G). These results suggest that the therapeutic effect of maternal BCAV on PN hypersensitivity in their offspring is associated with the alterations in IL-4 and IL-10. Given that production of the signature regulatory cytokine IL-10 by SPCs from BCAV fed mother's offspring was markedly increased, we next determined the number of CD4⁺CD25⁺T_{reg}s in the SPC culture and found an increase in T_{reg}% from BCAV fed mother's offspring as compared to sham treated mother's offspring and naïve offspring (FIG. 18H).

### Example 11. Cloning and Expression of CTB-Ara h8, CTB-Ara h6 and CTB-Ara h1-3 on Surfaces (Coats) of Spores

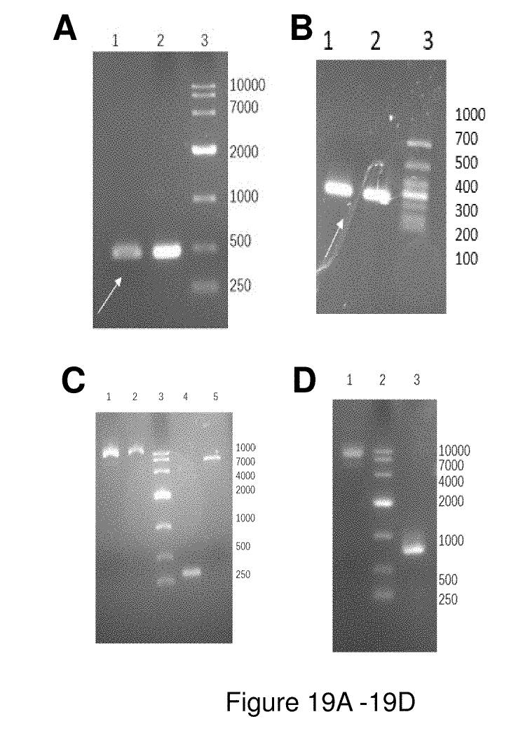

Recombinant plasmid of pus186-CotC-CTB-Ara h8 was transformed into B. subtilis and sporulation was formed in DSM using exhaustion method. PCR bands on gel electrophoresis were identified as Arah8 (FIG. 19A), CTB (FIG. 19B), CTB-Ara h8 clone in pET28a (FIG. 19C), and recombinant Pus186cotC-CTB-Arah8 plasmid (FIG. 19D).

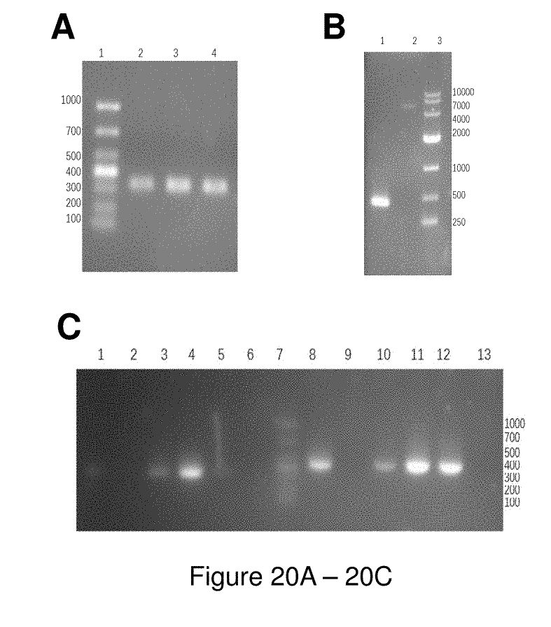

Recombinant plasmid of pus186-CotC-CTB-Ara h6 was transformed into B. subtilis and sporulation was formed in DSM using exhaustion method. PCR bands on gel electrophoresis were identified as CTB (FIG. 20A), recombinant pET28-CTB-Arah6 (FIG. 20B) and recombinant CTB-Ara6 in Pus186cotC-CTB-Ara h6 plasmid (FIG. 20C).

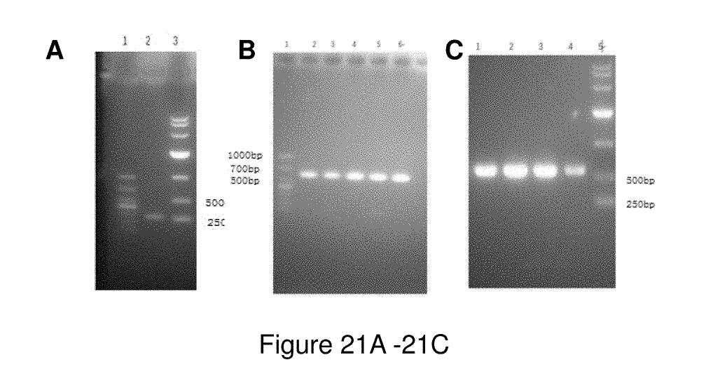

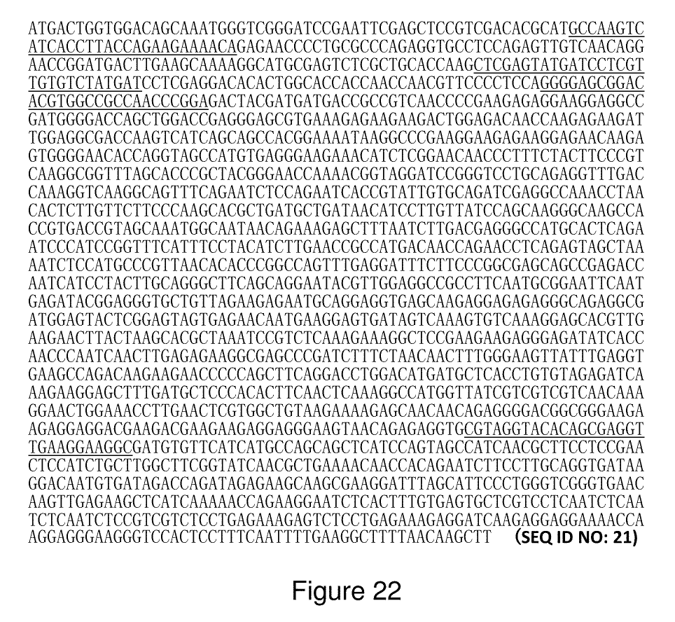

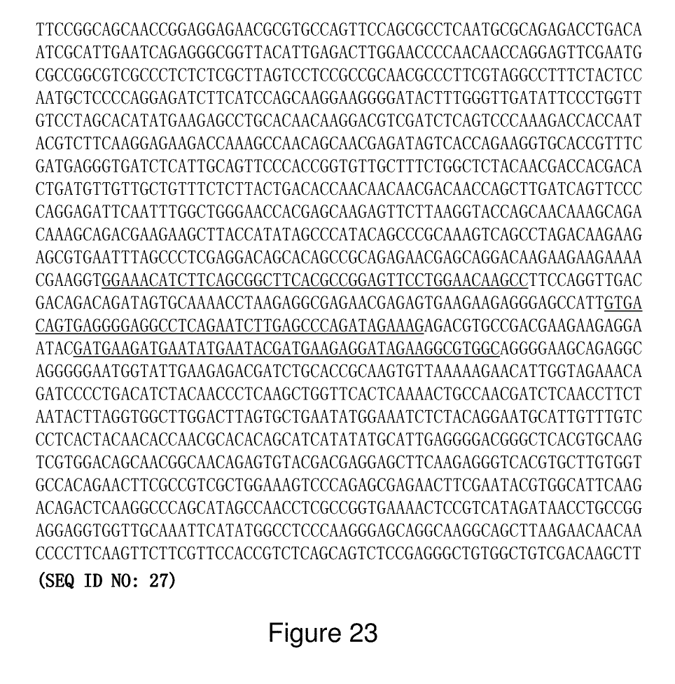

Recombinant plasmid of pus186-CotC-CTB-epitope Ara h1&3 was transformed into B. subtilis and sporulation was formed in DSM using exhaustion method, and PCR bands on gel electrophoresis were identified as Epitope Ara h1&3 (FIG. 21A), recombinant CTB-Epitope in pET28a-CTB-Epitope h1&3 (FIG. 21B) and recombinant CTB-Epitope h1&3 in Pus186cotC-CTB-Epitope 1&3 plasmid (FIG. 21C).

What is claimed is:

1. A composition comprising recombinant bacterial spores, wherein said recombinant bacterial spores express Cholera Toxin B (CTB) and an Ara h2 antigen on the surface of said spores.

2. The composition of claim 1, wherein said CTB and said Ara h2 antigen are co-expressed on the surface of the same spores among said recombinant bacterial spores.

3. The composition of claim 2, wherein said CTB and said Ara h2 antigen are expressed as a fusion protein.

4. The composition of claim 1, wherein said CTB and said Ara h2 antigen are expressed on the surfaces of different spores among said recombinant bacterial spores.

5. The composition of claim 1, wherein said Ara h2 antigen comprises an amino acid sequence substantially identical with SEQ ID NO: 90, or comprises at least one epitope selected from the group consisting of the epitopes listed in Table 2.

6. The composition of claim 1, wherein said recombinant bacterial spores also express an Ara h1 antigen.

7. The composition of claim 6, wherein said Ara h1 antigen is expressed on the cell surface of spores that also express either said CTB and/or said Ara h2 antigen.

8. The composition of claim 6, wherein said Ara h1 antigen comprises at least one epitope selected from the group consisting of the epitopes listed in Table 1.

9. The composition of claim 1, wherein said recombinant bacterial spores also express an Ara h3 antigen.

10. The composition of claim 9, wherein said Ara h3 antigen is expressed on the cell surface of spores that also express either said CTB and/or said Ara h2 antigen.

11. The composition of claim 9, wherein said Ara h3 antigen comprises at least one epitope selected from the group consisting of the epitopes listed in Table 3.

12. The composition of claim 1 wherein said recombinant bacterial spores also express an Ara h6 antigen.

13. The composition of claim 12, wherein said Ara h6 antigen is expressed on the cell surface of spores that also express either said CTB and/or said Ara h2 antigen.

14. The composition of claim 12, wherein said Ara h6 antigen comprises an amino acid sequence substantially identical with SEQ ID NO: 92, or comprises at least one epitope selected from the group consisting of the epitopes listed in Table 4.

15. The composition of claim 1, wherein said recombinant bacterial spores also express an Ara h8 antigen.

16. The composition of claim 15, wherein said Ara h8 antigen is expressed on the cell surface of spores that also express either said CTB and/or said Ara h2 antigen.

17. The composition of claim 15, wherein said Ara h8 antigen comprises an amino acid sequence substantially identical with SEQ ID NO: 93, or comprises at least one epitope selected from the group consisting of the epitopes listed in Table 5.

18. The composition of claim 1, wherein said recombinant bacterial spores also express at least one Ara h1 antigen and at least one Ara h3 antigen.

19. The composition of claim 1, wherein the recombinant bacterial spores are probiotic bacterial spores wherein the protiotic bacterial species are selected from the group consisting of Lactobacillus, Escherichia, Bacillus, Bifidobacterium, Saccharomyces and Streptococcus genera.

20. The composition of claim 1, wherein the recombinant bacterial spores are Bacillus subtilis spores.

21. A method of inducing tolerance to peanuts comprising administering an effective amount of the composition of claim 1 to a subject in need thereof.

22. The method of claim 21, wherein the effective amount of said composition comprises 1×10¹, 1×10², 1×10³, 1×10⁴, 1×10⁵, 1×10⁶, 1×10⁷, 1×10⁸, 1×10⁹ or more said recombinant bacterial spores.

23. The method of claim 21, wherein the composition is administered orally.

24. The method of claim 21, wherein the composition is administered with food.
